(19) United States
(12) Patent Application Publication
(10) Pub. No.: US 2024/0408057 A1
(43) Pub. Date: Dec. 12, 2024

(54) Composition for preventing or treating cachexia and muscle loss

(71) Applicant: METAFINES.CO.LTD., Seongnam-si (KR)

(72) Inventors: Chaul Min Pai, Seongnam-si (KR); Min Hyuk Yun, Yongin-si (KR); Young Jun Cho, Seongnam-si (KR); Hye Mi Cho, Seoul (KR)

(21) Appl. No.: 18/239,315

(22) Filed: Aug. 29, 2023

(30) Foreign Application Priority Data
Jun. 9, 2023 (KR) 10-2023-0073997

(51) Int. Cl. A61K 31/375; A61K 31/19; A61K 31/198; A61K 45/06; A61P 21/00; A61P 35/00

(52) U.S. Cl. CPC A61K 31/375; A61K 31/19; A61K 31/198; A61K 45/06; A61P 21/00; A61P 35/00

(57) ABSTRACT

The present disclosure provides a pharmaceutical composition and a food composition for preventing or treating cachexia and muscle loss, wherein the pharmaceutical composition and the food composition each comprise an ionic compound that comprises a calcium cation, a first anion, and a second anion, and wherein the first anion and the second anion are different from each other, and are each independently anions of ascorbic acid, dichloroacetic acid, a branched-chain amino acid (BCAA), or derivatives thereof.

### TECHNICAL FIELD

The present disclosure relates to a pharmaceutical composition for preventing or treating cachexia and muscle loss containing a calcium-based ionic compound as an active ingredient, and a food composition containing the active ingredient.

The present application claims priority to Korean Patent Application No. 10-2023-0073997 filed on Jun. 9, 2023 in the Republic of Korea, the disclosures of which are incorporated herein by reference.

### BACKGROUND

Cachexia is a complex syndrome caused by diseases such as cancer, tuberculosis, diabetes, AIDS, chronic obstructive pulmonary disease, multiple sclerosis, congestive heart failure, hemophilia, hypopituitarism, or liver cirrhosis. Cachexia does not recover with nutritional supplementation alone or has a low recovery rate, and causes loss of appetite or continuous muscle loss, leading to a state of weakness or death.

The prevalence of cachexia is approximately 10% in patients with heart failure, but is greater than 50% in cancer patients. It is known that cachexia symptoms appear in about 70% of gastrointestinal cancer patients, and cachexia symptoms appear in more than 80% of terminal cancer patients regardless of the type of cancer. Cancer treatment methods are categorized into surgical operation, radiation therapy, and chemotherapy. In the terminal stage when invasive treatment becomes difficult, applying radiation therapy or chemotherapy deteriorates the patient's nutritional status and immune functions, resulting in cachexia or the progression of the disease could accelerate. While the mortality rate of cachexia in patients with chronic obstructive pulmonary disease is approximately 20% and the mortality rate in patients with heart failure is approximately 30%, the mortality rate of cachexia in cancer patients reaches up to 80%, the importance of cachexia treatment has been drawing attention as much as the treatment of causative diseases.

Due to the exact mechanism of occurrence of cachexia has not been identified, conventional treatments for cachexia have involved increasing nutrient supply or appetite enhancers like megestrol acetate. However, these treatments had the problem in that the weight temporarily increased and then decreased again, or the physical ability was not improved despite the increase in weight, and in the case of a long-term use, these treatments had adverse effects such as erectile dysfunction, uterine bleeding, thromboembolism, edema, hyperglycemia, adrenal insufficiency, and hypertension. Accordingly, there is a need for a completely new approach to treatment based on understanding of the mechanisms of cachexia and muscle loss.

### SUMMARY

Considering the mechanism of occurrence and exacerbation of muscle loss, which is the main symptom of cachexia and based on the fact that cachexia is a multifactorial disease, an object of the present disclosure is to provide an active ingredient that acts in a multimodal manner and a composition for preventing or treating cachexia and muscle loss containing the active ingredient.

In addition, an object of the present disclosure is to provide a food composition containing the active ingredient.

An aspect of the present disclosure provides a pharmaceutical composition for preventing or treating cachexia and muscle loss containing an ionic compound, wherein the ionic compound comprises a calcium cation, a first anion, and a second anion, and wherein the first anion and the second anion are different from each other, and are each independently anions of ascorbic acid, dichloroacetic acid, a branched-chain amino acid (BCAA), or derivatives thereof.

The branched-chain amino acid may be one or more selected from the group consisting of leucine, isoleucine, and valine.

In the pharmaceutical composition may be administered such that the ionic compound is at a dose of 20 mg/kg/day to 4,800 mg/kg/day.

The pharmaceutical composition may further include a pharmaceutically acceptable aqueous solvent, wherein the degree of dissociation of the ionic compound may be 1% to 50%.

The pharmaceutical composition may further include one or more active agents selected from the group consisting of appetite enhancers, anticancer agents, anti-inflammatory agents, antibacterial agents, antifungal agents, antiviral agents, immunomodulators, steroids, anticoagulants, anticonvulsants, antidepressants, antioxidants, and vitamins.

The pharmaceutical composition may further include one or more selected from pharmaceutically acceptable carriers, excipients, and diluents.

The pharmaceutical composition may be formulated into a liquid, powder, an agent for oral administration, an injection, an infusion, an aerosol, a tablet, a capsule, a pill, a depot, or a suppository.

The pharmaceutical composition may be administered orally, intravenously, subcutaneously, intramuscularly, or transmucosally.

The cachexia and muscle loss may be caused by cancer, tuberculosis, diabetes, AIDS, chronic obstructive pulmonary disease, multiple sclerosis, congestive heart failure, hemophilia, hypopituitarism, or liver cirrhosis.

The cancer may be selected from the group consisting of lung cancer, breast cancer, colorectal cancer, stomach cancer, liver cancer, brain cancer, pancreatic cancer, thyroid cancer, skin cancer, bone marrow cancer, lymphoma, uterine cancer, cervical cancer, ovarian cancer, kidney cancer, and melanoma.

The muscle loss may be caused by sarcopenia, atony, muscular atrophy, muscular dystrophy, amyotrophic lateral sclerosis, or myasthenia gravis.

Another aspect of the present disclosure provides a food composition containing an ionic compound, wherein the ionic compound contains a calcium cation, a first anion, and a second anion, and wherein the first anion and the second anion are different from each other, and are each independently anions of ascorbic acid, dichloroacetic acid, a branched-chain amino acid (BCAA), or derivatives thereof.

The food composition may be a health functional food.

### Advantageous Effects

The composition according to the present disclosure provides the effect for preventing, improving, or treating cachexia and muscle loss that simultaneously inhibits, in a multi-modal method, (1) catabolism by energy metabolism, (2) disruption of muscle homeostasis, and (3) muscle loss by inflammatory factors.

According to the multimodal method, the composition can inhibit anorexia, maintain or increase body weight, reduce the amount of change in weight, reduce muscle loss or increase muscle mass, and improve muscle functions.

### BRIEF DESCRIPTION OF THE DRAWINGS

FIGS. 1A to 1C show the results of cytotoxicity tests of the calcium salt of Preparation Example 1 (FIG. 1A), the calcium salt of Preparation Example 2 (FIG. 1B), and the calcium salt of Preparation Example 3 (FIG. 1C) on cells before C2C12 differentiation.

FIGS. 2A to 2C show the results of cytotoxicity tests of the calcium salt of Preparation Example 1 (FIG. 2A), the calcium salt of Preparation Example 2 (FIG. 2B), and the calcium salt of Preparation Example 3 (FIG. 2C) on cells after C2C12 differentiation.

FIG. 3A shows a crystal violet staining image of the myotube of the normal control group (UCM), FIG. 3B shows a crystal violet staining image of a cancer cell culture medium treated group (CCM), and FIG. 3C shows the measurement results of the myotube diameter of FIGS. 3A and 3B with Image J.

FIG. 4 shows a Western blot result of markers, relating to degradation and differentiation of muscle, included in a normal control group (UCM) and a cancer cell culture medium treated group (CCM).

FIGS. 5A to 5D show graphs illustrating the relative expression levels of MuRF-1 (FIG. 5A), Atrogin-1 (FIG. 5B), MyHC (FIG. 5C), and Myogenin (FIG. 5D) from the Western blot results of FIG. 4.

FIGS. 6A to 6B show Western blot images (FIG. 6A) and a graph illustrating the relative expression levels of the target marker PDK-4 (FIG. 6B) according to the concentration of the calcium salt treatment in Preparation Example 1.

FIGS. 7A to 7B show Western blot images (FIG. 7A) and a graph illustrating the relative expression levels of the target marker Calpain 3 (FIG. 7B) according to the concentration of the calcium salt treatment in Preparation Example 1.

FIG. 8 shows the Western blot results of the target markers according to the concentration of the calcium salt treatment in Preparation Example 2.

FIGS. 9A to 9C show graphs illustrating the relative expression levels of target markers Calpain 3 (FIG. 9A), PDK-4 (FIG. 9B), and GDF-15 (FIG. 9C) from the Western blot results of FIG. 8.

FIG. 10 shows Western blot results of the target markers according to the concentration of the calcium salt treatment in Preparation Example 3.

FIGS. 11A to 11C show graphs illustrating the relative expression levels of target markers Calpain 3 (FIG. 11A), PDK-4 (FIG. 11B), and GDF-15 (FIG. 11C) from the Western blot results of FIG. 10.

FIGS. 12A to 12F show Western blot results (FIG. 12A) and a graph illustrating the relative expression level (FIG. 12B) of the target marker PDK-4; Western blot results (FIG. 12C) and a graph illustrating the relative expression level (FIG. 12D) of the target marker Calpain 3; and Western blot results (FIG. 12E) and a graph illustrating the relative expression level (FIG. 12F) of the target marker GDF-15, when myotube cells were treated with cancer cell culture medium (CCM).

FIG. 13A shows a crystal violet staining image of the C2C12 cell line differentiated into myotubes in a normal control group (UCM), FIG. 13B shows a crystal violet staining image of the WY-14643 (i.e., a PDK-4 activator) treated group (CCM), and FIG. 13C shows the measurement results of the myotube diameter of FIGS. 13A and 13B with Image J.

FIGS. 14A to 14B show Western blot results (FIG. 14A) and relative expression levels (FIG. 14B) of target and muscle-related markers (PDK-4, MuRF1, Atrogin1, MyHC, and Myogenin) in a normal control group (UCM) and the WY-14643 treated group used in FIG. 13.

FIG. 15A shows a crystal violet staining image of C2C12 cell line differentiated into myotubes in a normal control group (UCM), FIG. 15B shows an image of a group transfected with scrambled siRNA as a vehicle (excipient) control group, FIG. 15C shows an image of a group transfected with Calpain 3 siRNA, and FIG. 15D shows the measurement results of the myotube diameter of FIGS. 15A to 15C with Image J.

FIGS. 16A to 16B show Western blot results (FIG. 16A) and relative expression levels (FIG. 16B) of target and muscle-related markers (Calpain 3, MuRF1, Atrogin1, MyHC, and Myogenin) in a normal control group (UCM) and a vehicle control group (scrambled siRNA) and Calpain 3 siRNA treated group used in FIG. 15.

FIG. 17A shows a crystal violet staining images of C2C12 cell line differentiated into myotubes in a normal control group (UCM), a cancer cell culture medium treated group (CCM), and an experimental group in which CCM was treated with the calcium salt (Asc-Ca-DCA) of Preparation Example 1, and FIG. 17B shows the measurement results of the myotube diameter of FIG. 17A with Image J.

FIG. 18 shows Western blot results of the C2C12 cell line differentiated into myotubes in a normal control group (UCM), a cancer cell culture medium treated group (CCM), and an experimental group in which CCM was treated with the calcium salt (Asc-Ca-DCA) of Preparation Example 1.

FIGS. 19A to 19D show graphs illustrating the relative expression levels of muscle-related markers MuRF1 (FIG. 19A), Atrogin1 (FIG. 19B), MyHC (FIG. 19C), and Myogenin (FIG. 19D) from the Western blot results of FIG. 18.

FIG. 20 shows Western blot results of the C2C12 cell line differentiated into myotubes in a normal control group (UCM), a cancer cell culture medium treated group (CCM), and an experimental group in which CCM was treated with the calcium salt (Asc-Ca-Leu) of Preparation Example 2.

FIGS. 21A to 21D show graphs illustrating the relative expression levels of muscle-related markers MuRF1 (FIG. 21A), Atrogin1 (FIG. 21B), MyHC (FIG. 21C), and Myogenin (FIG. 21D) from the Western blot results of FIG. 20.

FIG. 22 shows Western blot results of the C2C12 cell line differentiated into myotubes in a normal control group (UCM), a cancer cell culture medium treated group (CCM), and an experimental group in which CCM was treated with the calcium salt (DCA-Ca-Leu) of Preparation Example 3.

FIGS. 23A to 23D show graphs illustrating the relative expression levels of muscle-related markers MuRF1 (FIG. 23A), Atrogin1 (FIG. 23B), MyHC (FIG. 23C), and Myogenin (FIG. 23D) from the Western blot results of FIG. 22.

FIG. 24A shows C2C12 cell line differentiated into myotubes crystal violet staining images of a normal control group (UCM), a cancer cell culture medium treated group (CCM), an experimental group in which CCM was treated with the calcium salt (Asc-Ca-DCA) of Preparation Example 1, an experimental group in which CCM was treated with ascorbic acid (Asc), an experimental group in which CCM was treated with dichloroacetic acid (DCA), and an experimental group in which CCM was treated with the combination of ascorbic acid (Asc) and dichloroacetic acid (DCA), and FIG. 24B shows the measurement results of the myotube diameter of each group in FIG. 24A with Image J.

FIG. 25 shows Western blot results for MuRF1, Atrogin1, MyHC, and Myogenin as muscle-related markers in the treatment groups of FIG. 24.

FIGS. 26A to 26D show graphs illustrating the relative expression levels of MuRF1 (FIG. 26A), Atrogin1 (FIG. 26B), MyHC (FIG. 26C), and Myogenin (FIG. 26D) from the Western blot results of FIG. 25.

FIG. 27 shows Western blot results for Calpain 3, PDK-4, and GDF-15 as target markers in the treatment groups of FIG. 24.

FIGS. 28A to 28C show graphs illustrating the relative expression levels of Calpain 3 (FIG. 28A), PDK-4 (FIG. 28B), and GDF-15 (FIG. 28C) from the Western blot results of FIG. 27.

FIG. 29 shows Western blot results for MuRF1, Atrogin1, MyHC, and Myogenin as muscle-related markers in a normal control group (UCM), a cancer cell culture medium treated group (CCM), and an experimental group in which CCM was treated with the calcium salt (Asc-Ca-Leu) of Preparation Example 2, an experimental group in which CCM was treated with ascorbic acid (Asc), an experimental group in which CCM was treated with leucine (Leu), and an experimental group in which CCM was treated with the combination of ascorbic acid (Asc) and leucine (Leu).

FIGS. 30A to 30D show graphs illustrating the relative expression levels of MuRF1 (FIG. 30A), Atrogin1 (FIG. 30B), MyHC (FIG. 30C), and Myogenin (FIG. 30D) from the Western blot results of FIG. 29.

FIG. 31 shows Western blot results for Calpain 3, PDK-4, and GDF-15 as target markers in a normal control group (UCM), a cancer cell culture medium treated group (CCM), and an experimental group in which CCM was treated with the calcium salt (Asc-Ca-Leu) of Preparation Example 2, an experimental group in which CCM was treated with ascorbic acid (Asc), an experimental group in which CCM was treated with leucine (Leu), and an experimental group in which CCM was treated with the combination of ascorbic acid (Asc) and leucine (Leu).

FIGS. 32A to 32C show graphs illustrating the relative expression levels of Calpain 3 (FIG. 32A), PDK-4 (FIG. 32B), and GDF-15 (FIG. 32C) from the Western blot results of FIG. 31.

FIG. 33 shows Western blot results for MuRF1, Atrogin1, MyHC, and Myogenin as muscle-related markers in a normal control group (UCM), a cancer cell culture medium treated group (CCM), and an experimental group in which CCM was treated with the calcium salt (DCA-Ca-Leu) of Preparation Example 3, an experimental group in which CCM was treated with dichloroacetic acid (DCA), an experimental group in which CCM was treated with leucine (Leu), and an experimental group in which CCM was treated with the combination of dichloroacetic acid (DCA) and leucine (Leu).

FIGS. 34A to 34D show graphs illustrating the relative expression levels of MuRF1 (FIG. 34A), Atrogin1 (FIG. 34B), MyHC (FIG. 34C), and Myogenin (FIG. 34D) from the Western blot results of FIG. 33.

FIG. 35 shows Western blot results for Calpain 3, PDK-4, and GDF-15 as target markers in a normal control group (UCM), a cancer cell culture medium treated group (CCM), and an experimental group in which CCM was treated with the calcium salt (DCA-Ca-Leu) of Preparation Example 3, an experimental group in which CCM was treated with dichloroacetic acid (DCA), an experimental group in which CCM was treated with leucine (Leu), and an experimental group in which CCM was treated with the combination of dichloroacetic acid (DCA) and leucine (Leu).

FIGS. 36A to 36C show graphs illustrating the relative expression levels of Calpain 3 (FIG. 36A), PDK-4 (FIG. 36B), and GDF-15 (FIG. 36C) from the Western blot results of FIG. 35.

FIG. 37A shows crystal violet staining images of myotube in a normal control group (UCM), a cancer cell culture medium treated group (CCM), and an experimental group in which UCM was treated with dexamethasone (Dex); and FIG. 37B shows the measurement results of the myotube diameter of FIG. 37A with Image J.

FIG. 38 shows Western blot results for MuRF1, Atrogin1, MyHC, Myogenin, Calpain 3, PDK-4, and GDF-15 as target and muscle-related markers in a normal control group (UCM), a cancer cell culture medium treated group (CCM), and an experimental group in which UCM was treated with dexamethasone (Dex).

FIG. 39 shows Western blot results for MuRF1, Atrogin1, MyHC, Myogenin, Calpain 3, PDK-4, and GDF-15 as target and muscle-related markers in a normal control group (UCM), an experimental group in which UCM was treated with dexamethasone (Dex), and an experimental group in which UCM was treated with the calcium salt (Asc-Ca-DCA) of Preparation Example 1.

FIGS. 40A to 40G show graphs illustrating the relative expression levels of MuRF1 (FIG. 40A), Atrogin1 (FIG. 40B), MyHC (FIG. 40C), Myogenin (FIG. 40D), Calpain 3 (FIG. 40E), PDK-4 (FIG. 40F), and GDF-15 (FIG. 40G) from the Western blot results of FIG. 39.

FIG. 41 shows Western blot results for MuRF1, Atrogin1, MyHC, Myogenin, Calpain 3, PDK-4, and GDF-15 as target and muscle-related markers in a normal control group (UCM), an experimental group in which UCM was treated with dexamethasone (Dex), and an experimental group in which UCM was treated with the calcium salt (Asc-Ca-DCA) of Preparation Example 2.

FIGS. 42A to 42G show graphs illustrating the relative expression levels of MuRF1 (FIG. 42A), Atrogin1 (FIG. 42B), MyHC (FIG. 42C), Myogenin (FIG. 42D), Calpain 3 (FIG. 42E). PDK-4 (FIG. 42F), and GDF-15 (FIG. 42G) from the Western blot results of FIG. 41.

FIG. 43 shows Western blot results for MuRF1, Atrogin1, MyHC, Myogenin, Calpain 3, PDK-4, and GDF-15 as target and muscle-related markers in a normal control group (UCM), an experimental group in which UCM was treated with Dex, and an experimental group in which UCM was treated with the calcium salt (DCA-Ca-Leu) of Preparation Example 3.

FIGS. 44A to 44G show graphs illustrating the relative expression levels of MuRF1 (FIG. 44A), Atrogin1 (FIG. 44B), MyHC (FIG. 44C), Myogenin (FIG. 44D), Calpain 3 (FIG. 44E), PDK-4 (FIG. 44F), and GDF-15 (FIG. 44G) from the Western blot results of FIG. 43.

In FIGS. 45 to 53 below, each group represents the following. G1: normal control group, G2: the a vehicle (excipient) administration group, G3: megestrol administration group (125 mpk), G4: calcium salt of Preparation Example 1 oral administration group (200 mpk), G5: the calcium salt of Preparation Example 1 (100 mpk) intravenous administration group, and G6: the calcium salt of Preparation Example 1 (200 mpk) intravenous administration group.

FIG. 45 shows Western blot results for MuRF1, Atrogin1, MyHC, and Myogenin as muscle-related markers in mouse muscle tissue.

FIGS. 46A to 46D show graphs illustrating the relative expression levels of MuRF1 (FIG. 46A), Atrogin1 (FIG. 46B), MyHC (FIG. 46C), and Myogenin (FIG. 46D) from the Western blot results of FIG. 45.

FIGS. 47A to 47C show Western blot results for Calpain 3 and PDK-4 as target markers in mouse muscle tissue (FIG. 47A), and graphs illustrating the relative expression levels of Calpain 3 (FIG. 47B) and PDK-4 (FIG. 47C) from the Western blot results above.

FIGS. 48A to 48B show graphs illustrating the concentrations of IL-1β (FIG. 48A) and lactate (FIG. 48B) measured in mouse blood.

FIGS. 49A to 49B show graphs illustrating the effect of inhibiting weight loss in mice according to time lapse (FIG. 49A) and experimental groups (FIG. 49B).

FIGS. 50A to 50F show images illustrating cross-sections of the gastrocnemius of mice in each of experimental groups G1 to G6, and FIG. 50G shows a graph summarizing the cross-sectional areas from FIGS. 50A to 50F.

FIGS. 51A to 51F show images illustrating cross-sections of quadriceps of mice in each of experimental groups G1 to G6, and FIG. 51G shows a graph summarizing the cross-sectional areas from FIGS. 51A to 51F.

FIGS. 52A to 52F show images illustrating cross-sections of the soleus of mice in each of experimental groups G1 to G6, and FIG. 52G shows a graph summarizing the cross-sectional areas from FIGS. 52A to 52F.

FIG. 53A shows the result of the Rotarod-treadmill test in mice, and FIG. 53B shows the result of the wire hanging test in mice.

### DETAILED DESCRIPTION

Hereinafter, each component of the present disclosure will be described in more detail so that those skilled in the art to which the present disclosure belongs can easily practice the invention, but this is only an example, and the scope of rights of the present disclosure is not limited by the following described herein below.

As used herein, the term “include” is used when listing materials, compositions, devices, and methods useful in the present disclosure, and is not limited to the examples listed.

As used herein, “about” and “substantially” are used to mean a numerical value or range of degrees or approximations thereto, taking into account of an inherent manufacture and material tolerances, and are used to indicate the exact or absolute meaning provided to aid the understanding of the present disclosure. Numerical values are used to prevent the unfair use by an infringer of the disclosed content.

As used herein, the term “subject” may refer to a mammal exhibiting any one or more symptoms from cachexia and muscle loss.

As used herein, the term “treatment” refers to any action that improves or benefits one or more symptoms of cachexia and muscle loss by administering a composition according to a specific embodiment of the present disclosure to a subject.

In a specific embodiment of the present disclosure provides a pharmaceutical composition for preventing or treating cachexia and muscle loss comprising an ionic compound, wherein the ionic compound includes a calcium cation, a first anion, and a second anion, and wherein the first anion and the second anion are different from each other, and are each independently anions of ascorbic acid, dichloroacetic acid, a branched-chain amino acid (BCAA), or derivatives thereof.

The ionic compound refers to a compound in which ions having opposite charges form an ionic bond by an electrostatic force, and may exhibit electrical neutrality. Specifically, the ionic compound may be one in which two anions, which are selected from the group consisting anions of ascorbic acid, dichloroacetic acid, branched-chain amino acids, and derivatives thereof, are ionically bonded to both sides of a Ca²⁺ ion (calcium ion). Preferably, the ionic compound may be one in which two mutually-different anions selected from anions of ascorbic acid, dichloroacetic acid, and branched-chain amino acid are ionically bonded to both sides of the calcium ion. For example, the ionic compound may include a calcium salt of ascorbic acid and dichloroacetic acid; a calcium salt of ascorbic acid and a branched-chain amino acid; a calcium salt of dichloroacetic acid and a branched-chain amino acid.

The branched-chain amino acid (BCAA) may include one or more selected from the group consisting of leucine, isoleucine, and valine. The branched-chain amino acid can contribute to the synthesis and conversion of proteins. Preferably, the branched-chain amino acid may be leucine. For example, the ionic compound may include a calcium salt of ascorbic acid and leucine; and a calcium salt of dichloroacetic acid and leucine.

The ionic compound simultaneously provides the following three mechanisms relating to cachexia and muscle loss, thereby providing the effects of prevention, improvement, or treatment of cachexia and muscle loss, which are multifactorial diseases:

(1) The pharmaceutical composition containing the ionic compound inhibits catabolism caused by energy metabolism, thereby preventing weight loss due to unnecessary energy consumption.

For example, in the case of cancer cachexia and muscle loss, cancer cells can perform energy metabolism through oxidative phosphorylation that requires oxygen and glycolysis that does not use oxygen. In particular, cancer cells mainly utilize glycolysis to survive even under the hypoxia environment in which normal cells cannot survive, and the apoptosis control process starting from mitochondria is inactivated. When the glycolysis of cancer cells is increased, glycogen is broken down into lactic acid in the liver. Lactic acid can be converted to pyruvic acid by lactate dehydrogenase (LDH), and pyruvic acid is further converted to oxaloacetate and malate, and finally, phosphoenolpyruvic acid (PEP) by phosphoenolpyruvate carboxyl kinase. Fructose-1,6-biphosphate is produced from phosphoenolpyruvic acid, and fructose-1,6-biphosphate is converted to fructose-6-phosphate and is then converted to glucose via glucose-6-phosphate. This series of processes is referred to as gluconeogenesis, and a total of 4 ATP and 2 GTP are required to generate one molecule of glucose. As a result, the abnormal anaerobic glycolysis in cancer cells induces the enhancement of gluconeogenesis, which leads to waste of unnecessary energy in the body thereby causing weight loss.

L-ascorbic acid (Asc) included in the ionic compound is a substance widely known as vitamin C, and was used for prevention and treatment of scurvy in the early days of discovery; however, as other functions (e.g., strong antioxidant power and involvement in the production of specific neurotransmitters) have been revealed, they have been used in various fields (e.g., pharmaceuticals, foods, and cosmetics). Since ascorbic acid has excellent electron donating ability, it has excellent antioxidant action, removal of active oxygen, and anti-aging abilities, and can act as a coenzyme for various enzymes in the body. In particular, ascorbic acid can act as the coenzyme for an enzyme called prolyl hydroxylase. Prolyl hydroxylase can be activated by ascorbic acid to thereby hydroxylate and remove the proline residue of HIF-1α protein. HIF-1α protein is a subunit constituting hypoxia inducible factor-1 (HIF-1), which is a heterodimer, and is involved in the control of energy metabolism and angiogenesis. Since cancer cells consume excessive energy and nearby oxygen, they require oxygen and energy source supply through the angiogenic function of HIF-1α protein. Therefore, the decrease of HIF-1α protein following the administration of ascorbic acid can provide an inhibitory effect on tumor growth by creating the unfavorable environment for the growth of cancer cells, and simultaneously can prevent weight loss by inhibiting the excessive gluconeogenesis and reducing extraordinal energy consumption.

Dichloroacetic acid (DCA) included in the ionic compound is known to have a therapeutic effect on metabolic diseases caused by mitochondrial abnormalities when provided in the form of a salt. Specifically, cancer cells are in a state where pyruvate is not converted to acetyl-CoA because HIF-1, which is a hypoxic inducer, enhances the expression of pyruvate dehydrogenase kinase (PDK). Sodium dichloroacetic acid can activate oxidative phosphorylation in mitochondria by increasing the activity of pyruvate dehydrogenase (PDH) through inhibition of pyruvate dehydrogenase kinase (PDK) brings to convert pyruvate to acetyl-CoA. The provision of dichloroacetic acid salt through administration of the ionic compound not only inhibits lactic acid release and liver gluconeogenesis by inhibiting glycolysis through oxidative phosphorylation activity of cancer cells in mitochondria, but also can induce apoptotic cell death of cancer cells. This process may be referred to as mitochondrial reforming, which allows mitochondria to perform their normoxia metabolism in cancer cells. The ionic compound inhibits growth of cancer cells through mitochondrial reforming and changes the surrounding environment of cancer cells to reduce blood glucose level and lactic acid concentration, and inhibit excessive gluconeogenesis and, thereby inhibiting weight loss.

As a kind of branched-chain amino acid included in the ionic compound, leucine (Leu) is a kind of essential amino acid used in protein biosynthesis, and is not synthesized in the body; therefore, it must be supplied through the diet. Leucine is known to be involved in synthesis of muscle proteins including skeletal muscle, and acts as an important regulatory factor in initiating the translation during protein synthesis. In particular, leucine can act on the insulin signaling pathway. Specifically, leucine can act on pancreatic cells to directly promote insulin secretion or reduce blood glucose level by inhibiting glucagon secretion. Insulin can activate insulin receptors and activate Akt signaling proteins through the insulin signaling pathway. The activated Akt activates the TSC1/2 complex, and the TSC1/2 complex can activate mTORC1 (mammalian target of rapamycin complex 1), which controls protein synthesis, thereby activating protein synthesis. When insulin resistance occurs, the insulin signaling pathway cannot sufficiently activate the Akt signaling protein, thereby resulting in inactivation of mTORC1. Together with insulin, leucine is involved in mTORC1 activity, thereby regulating blood glucose level and simultaneously contributing to protein synthesis. mTORC1 interacts with metabolism-related proteins (e.g., AMPK.), leucine can induce the transmembrane movement of GLUT4 by involving in the activation of AMPK and help glucose uptake into cells. The ionic compound can activate the mechanism of reducing blood glucose levels through the supply of leucine and contribute to protein synthesis, thereby inhibiting weight loss.

The calcium ions included in the ionic compound may induce cancer cell death by affecting calcium homeostasis of cancer cells. Specifically, according to the administration of the pharmaceutical composition, calcium is accumulated in the mitochondria of cancer cells, and an excessive amount of reactive oxygen species (ROS) is generated in cancer cells, thereby resulting in apoptotic cell death caused by apoptosis. In addition, calcium ions can form a direct bond to α-ketoglutarate dehydrogenase complex (α-KGDC) and act as a regulator for the TCA cycle. The ionic compound excessively increases the calcium ion concentration in cancer cells through the supply of calcium, this can induce mitochondrial metabolic disorders through the activation of endonuclease and protease to thereby promote the release of cytochrome c. The release of cytochrome c activates caspase-9, and caspase-3 and caspase-7 are also activated, thereby resulting in apoptosis of cancer cells.

The pharmaceutical composition containing the ionic compound may act as a multimodal agent for prevention or treatment of cachexia and muscle loss by the combined contribution. Each of calcium ion, the first anion, and the second anion selected from the anions described above contribute to apoptosis of cancer cells, reduction of unnecessary energy consumption, activation of the mechanism of reducing blood glucose level and lactic acid concentration, and activation of protein synthesis.

The effect of the treatment of the pharmaceutical composition containing the ionic compound can be evaluated by checking the expression level of PDK as a target marker. Specifically, when the pharmaceutical composition containing the ionic compound is administered, it may appear that the expression level of PDK-4 (PDK4) decreases in tumor and muscle tissues of the subject suffering from cachexia or muscle loss. A decrease in the expression level of PDK-4 means activation of oxidative phosphorylation in mitochondria, and may include a decrease in unnecessary metabolism in the body and a decrease in blood glucose level and lactic acid concentration.

(2) The pharmaceutical composition containing the ionic compound can maintain muscle homeostasis and prevent a decrease in muscle mass.

A decrease in muscle mass refers to a decrease in the size of muscle cells and muscle tissue that occurs under various catabolic conditions caused by abnormality of related genes, hormonal imbalance, severe injury, sepsis, cancer, and aging.

The ascorbic acid included in the ionic compound can reduce the expression level of growth differentiation factor 15 (GDF-15), which is a cachexia-related index. GDF-15 is known as a marker relating to obesity and diabetes; however, it has recently been revealed that cachexia patients have higher levels of GDF-15 than healthy people in clinical practice, and the expression of GDF-15 is attracting attention as one being related to cachexia. Since HIF-1 can increase the GDF-15 level, the reduction of HIF-1 by administration of ascorbic acid can reduce the expression level of GDF-15, thereby providing effects for preventing or treating cachexia. In addition, the dichloroacetic acid included in the ionic compound can reform the mitochondrial mechanism and reduce the expression level of GDF-15, and when administered together with ascorbic acid, it can induce a synergistic effect for preventing or treating cachexia.

The dichloroacetic acid included in the ionic compound can inhibit the gluconeogenesis process by reducing blood glucose level and lactic acid concentration. The exact mechanism of muscle mass loss is not known, but the Cori-cycle is thought to be involved. When anaerobic glucose degradation occurs in muscle cells, lactic acid can be produced through glycolysis, and lactic acid moves to the liver and glucose is produced by gluconeogenesis, which is then supplied to muscle cells and tumor, and used as an energy source. As described above, the process of gluconeogenesis causes unnecessary energy waste and muscle loss. The dichloroacetic acid salt can also inhibit the mechanism of the Cory-cycle, thereby providing an effect of preventing muscle loss. In muscle tissue and myotubes of animals exhibiting reduced muscle mass, PDK-4 may be overexpressed compared to normal tissues and cells. The dichloroacetic acid salt can reduce the expression level of overexpressed PDK-4, which specifically appears in muscle during muscle loss, along with the metabolic control function described above, thereby providing effects for preventing or treating muscle loss.

In addition, as described above, it is known that there is a correlation between muscle reduction and the expression level of GDF-15, and it is confirmed that mitochondrial dysfunction acts as a major mechanism mediating the correlation. Mitochondrial dysfunction can be caused by a defect in the internal electron transport system or mitochondrial damage by oxidative stress. As the inflammatory response caused by oxidative stress increases, the expression level of GDF-15 may increase. The dichloroacetic acid salt can reduce the expression level of GDF-15 by improving mitochondrial dysfunction through mitochondrial reforming. Specifically, PGC-1α, as a transcription factor involved in mitochondrial function, regulates cellular energy metabolism, and the dichloroacetic acid salt can affect the PGC-1α and activate AMP-activated protein kinase (AMPK), which is mechanistically related to the PGC-1α, thereby regulating the energy state and metabolism within the cell. As such, the dichloroacetic acid salt affects the mitochondrial reforming mechanism, which are associated with PDK and PDH, and factors acting on energy metabolism and mitochondrial function, thereby provide combined contribution to the reforming of mitochondrial function, and through this, it can provide an effect for preventing or treating diseases relating to cachexia and muscle loss by reducing the expression level of GDF-15.

Leucine, as a kind of branched-chain amino acid included in the ionic compound, is involved in the IGF-1/PI3K/Akt mechanism relating to muscle synthesis, and thereby can provide the effects of protein synthesis and cell growth. Insulin-like growth factor-1 (IGF-1) is a hormone having a molecular structure similar to insulin, and is involved in the growth, maintenance, and metabolism of the body. IGF-1 contributes to mTORC1 activation through the PI3K/Akt signaling pathway. Leucine can activate mTORC1 through the insulin transport pathway and the IGF/PI3K/Akt mechanism, thereby reducing blood glucose level and activating protein synthesis.

The calcium ions included in the ionic compound can improve the structure and formation of muscle by increasing the expression level of calpain 3 which is related to muscle formation. Calpain 3 is a kind of non-lysosomal cysteine protease that is calcium-dependent and is known to cause muscle loss when its expression level is low. Calpain 3 controls the structure of proteins in tissues or fibroblasts and plays an important role in the growth and maintaining the shape of muscle fibers. Genetic mutations related to calpain 3 cause a deficiency of dystrophin, which is a protein in muscle fibers, thereby causing muscular diseases, such as Duchenne muscular dystrophy in which muscle strength and exercise ability are lost, and limb girdle muscular dystrophy (LGMD) in which muscle strength is weakened by degeneration of muscle tissue and damage to muscle fibers. The ionic compound increases through the supply of calcium the expression level of calpain 3, which is a marker that directly affects not only cachexia and muscle loss but also muscle-related diseases, thereby providing the effect of preventing or treating these diseases. In addition, leucine included in the ionic compound can increase the expression of calpain 3 through activation of mTORC1, and when administered together with calcium ions, it may cause a synergistic effect on the prevention or treatment of cachexia and muscle loss.

The pharmaceutical composition containing the ionic compound may act as a multimodal agent for prevention or treatment of cachexia and muscle loss by the combined contribution, of calcium ions and the first anion and the second anion selected from the anions described above, to one or more of regulation of expression level of markers related to muscle loss, decrease of blood glucose level, and protein synthesis.

The effect of the treatment of the pharmaceutical composition containing the ionic compound can be evaluated by checking the expression level of one of GDF-15 expression level, PDK-4 expression level, and calpain 3 expression level.

Specifically, when a pharmaceutical composition containing the ionic compound is administered, the expression level of GDF-15 in subjects suffering from cachexia or muscle loss may be reduced. GDF-15 is known to transmit a signal to the nervous system through a receptor called GDNF Family Receptor Alpha-Like (GFRAL) to express a mechanism that suppresses appetite and controls body weight. The appetite suppression mechanism of GDF-15-GFRAL can be divided into the following three steps: first, expression of GDF-15 is increased due to the mitochondrial dysfunction described above; second, the increased GDF-15 specifically binds to GFRAL and transmits a signal to a specific region of the brainstem including nucleus tractus solitarius (NTS); and third, various neurotransmitters are regulated and appetite is suppressed in the brainstem that has received the signal. The ascorbic acid and/or dichloroacetic acid included in the ionic compound can provide a fundamental therapeutic effect on appetite suppression by reducing the expression level of GDF-15, which is the first step as the starting point of the above mechanism.

Specifically, when the pharmaceutical composition containing the ionic compound is administered, the expression level of PDK-4 may be reduced in subjects suffering from cachexia or muscle loss.

Specifically, when the pharmaceutical composition containing the ionic compound is administered, the expression level of calpain 3 may be increased in subjects suffering from cachexia or muscle loss. Calpain 3 may affect mechanisms involved in calcium-induced breakdown of muscle proteins, and it may affect muscle loss by influencing c-FLIP (Caspase-8 Long Isoform), a protein involved in the process of apoptosis. Calpain 3 is expressed in muscle cells and inhibits the degradation of c-FLIP, thereby inhibiting the function of caspase-8. The inhibition of caspase-8 leads to inhibition of apoptosis, thereby preventing muscle loss and maintaining structural stability of muscle cells. Calpain 3 can prevent muscle loss through interaction with myostatin, which is a protein involved in muscle loss, as well as c-FLIP. The ionic compound can provide an effect for preventing muscle loss through an increase in the expression level of calpain 3.

(3) The pharmaceutical composition containing the ionic compound can prevent a decrease in muscle mass caused by inflammatory factors.

Inflammatory cytokines can be identified regardless of the type of cancer and are known to be a major cause of muscle loss. Specifically, inflammatory factors such as tumor necrosis factor-α (TNF-α), interleukin-1β (IL-1β), and interleukin-6 (IL-6) can induce cachexia and muscle loss. For example, a tumor necrosis factor can activate the ubiquitin-proteasome system (UPS), which degrades proteins present in the cytoplasm, promote breakdown of muscle proteins, damage muscle cells by increasing oxides, or reduce the production and regeneration of muscle proteins by reducing the expression of stress proteins.

The ascorbic acid included in the ionic compound can inhibit the secretion of inflammatory cytokines, simultaneously play the role of a low-toxic metabolizing agent that directly inhibits cancer cell growth, and can provide effects for preventing or treating cachexia and muscle loss caused by inflammatory factors. Ascorbic acid can inhibit cytokine secretion by inhibiting NF-κB and COX2, which are representative factors that mediate and regulate inflammatory responses. Specifically, ascorbic acid can suppress cytokine secretion by inhibiting the NF-κB signaling pathway. The inhibition of the NF-κB signaling pathway by ascorbic acid can also inhibit the mechanism of GDF-15, thereby preventing muscle loss.

Cyclooxygenase (COX) catalyzes the conversion of prostaglandin, which is a major mediator of inflammatory reactions, into arachidonic acid. COX1 is expressed at a constant level in the body to produce prostaglandins essential for the expansion of blood vessels, maintenance of blood flow, and protection of the gastric mucosa. In contrast, COX2 is rarely expressed in normal tissues and is induced by inflammatory factors, cell growth factors, tumor promoting factors, etc., and instantaneously produces a large amount of prostaglandin, thereby inducing various inflammatory reactions. While COX2 is overexpressed in cancer cells to promote cancer cell proliferation and metastasis and inhibit apoptosis, ascorbic acid can inhibit the mechanism of COX2 to prevent exacerbation of cachexia and muscle loss.

The dichloroacetic acid included in the ionic compound can reduce the release of lactic acid in the body, thereby reducing the expression of cytokines. Lactic acid can regulate signaling pathways that regulate the expression of several enzymes involved in cytokines and other immune responses through the activation of NF-κB and HIF-1. The ionic compound supplies dichloroacetic acid to inhibit the mechanism of cytokines by reducing the release of lactic acid, thereby preventing a decrease in protein production.

Leucine, as a kind of branched-chain amino acid included in the ionic compound, can inhibit the secretion of inflammatory cytokines (i.e., tumor necrosis factor, IL-6, and IFNγ), thereby preventing muscle loss. Leucine can inhibit the phosphorylation level of NF-κB, thereby inhibiting the secretion of cytokines. The ionic compound can supply leucine to inhibit the mechanism of cytokines, thereby preventing the decrease of protein production.

In the pharmaceutical composition containing the ionic compound, a first anion and a second anion can inhibit secretion of inflammatory cytokines, and thus can act as a multimodal agent for preventing or treating cachexia and muscle loss.

The effect of the treatment of the pharmaceutical composition containing the ionic compound can be evaluated by checking one or more of the expression level of GDF-15, as a target marker, the expression levels of cytokines, and the amount of lactate in the blood. Specifically, when the pharmaceutical composition containing the ionic compound is administered, the expression level of GDF-15 in subjects suffering from cachexia or muscle loss may be reduced. When the pharmaceutical composition containing the ionic compound is administered, the expression level of cytokines may be reduced in subjects suffering from cachexia or muscle loss. When the pharmaceutical composition containing the ionic compound is administered, the amount of lactic acid in the blood may be reduced in subjects suffering from cachexia or muscle loss.

The ionic compound may be provided at a dose of 20 mg/kg/day to 4,800 mg/kg/day. Specifically, the dose of the ionic compound may be 500 mg/kg/day or more and 4,500 mg/kg/day or less, 1,000 mg/kg/day or more and 4,000 mg/kg/day or less, 1,500 mg/kg/day or more and 3,500 mg/kg/day or less, or 2,000 mg/kg/day or more and 3,000 mg/kg/day or less. Preferably, the dose of the ionic compound may be 20 mg/kg/day or more and 2,000 mg/kg/day or less. Effective preventive or therapeutic effects on cachexia and muscle loss can be provided without cytotoxicity within the above-described dose range of the ionic compound. Within the above range, a more specific dose of the ionic compound may be determined depending on the type of the ionic compound, the subject's age, weight, health, sex, sensitivity to the ionic compound, administration time of the pharmaceutical composition, administration route, discharge rate, duration of treatment, or type and quantity of drugs used simultaneously.

The frequency of administration of the ionic compound may be once daily, but is not limited thereto. The ionic compound may be administered multiple times by dividing the dose within the above range.

The pharmaceutical composition may further include the ionic compound and a pharmaceutically acceptable aqueous solvent. The aqueous solvent may be distilled water, a saline solution, an injection solution or buffer solution, but is not limited thereto.

For example, the pharmaceutical composition may be a composition for oral administration, and may be prepared by adding 20 mg to 4,800 mg of the ionic compound to 1 mL of distilled water.

In a specific embodiment, the pharmaceutical composition for oral administration may be prepared by adding 160 mg of an ionic compound containing a calcium cation, an ascorbic acid anion, and a dichloroacetic acid anion to 1 mL of distilled water.

In a specific embodiment, the pharmaceutical composition for oral administration may be prepared by adding 200 mg of an ionic compound containing a calcium cation, an ascorbic acid anion, and a branched-chain amino acid anion to 1 mL of distilled water.

In a specific embodiment, the pharmaceutical composition for oral administration may be prepared by adding 480 mg of an ionic compound containing a calcium cation, a dichloroacetic acid anion, and a branched-chain amino acid anion to 1 mL of distilled water.

For example, the pharmaceutical composition may be a composition for intravenous administration, and may be prepared by injecting 20 mg to 4,800 mg of the ionic compound into 1 mL of an injection solution.

In a specific embodiment, the pharmaceutical composition for intravenous administration may be prepared by adding 80 mg of an ionic compound containing a calcium cation, an ascorbic acid anion, and a dichloroacetic acid anion into 1 mL of an injection solution.

In a specific embodiment, the pharmaceutical composition for intravenous administration may be prepared by adding 100 mg of an ionic compound containing a calcium cation, an ascorbic acid anion, and a branched-chain amino acid anion to 1 mL of an injection solution.

In a specific embodiment, the pharmaceutical composition for oral administration may be prepared by adding 240 mg of an ionic compound containing a calcium cation, a dichloroacetic acid anion, and a branched-chain amino acid anion to 1 mL of an injection solution.

The ionic compound may be mixed with the aqueous solvent to dissociate at least a portion of ionic bonds. The degree of dissociation of the ionic compound may be 1% to 50%. The ionic compound can maintain its chemical structure without being dissociated at least 1% or more in the pharmaceutical composition, and even after administration to a subject, the chemical structure can be maintained thereby being capable of providing the multimodal effect described above. The pharmaceutical composition containing the ionic compound may include an ionic compound dissociated in the aqueous solvent and an ionic compound dispersed in the aqueous solvent without being dissociated. The pharmaceutical composition can have an excellent effect for preventing or treating cachexia and muscle loss, compared to an aqueous solution containing the same contents of cations and anions that can be included in the ionic compound of the present disclosure.

The pharmaceutical composition may include the ionic compound and one or more pharmaceutically acceptable active agents. Specifically, the active agent may include appetite enhancers, anticancer agents, anti-inflammatory agents, antibacterial agents, antifungal agents, antiviral agents, immunomodulators, steroids, anticoagulants, anticonvulsants, antidepressants, antioxidants, and vitamins, but is not limited thereto. That is, the ionic compound may be used in combination with the active agents.

For example, the appetite enhancer may be megestrol, cyproheptadine, dronabinol, or metoclopramide, but is not limited thereto.

For example, the anticancer agent may be imatinib, 5-florouracil (5-FU), irinotecan, sunitinib, oxaliplatin, cisplatin, paclitaxel, lapatinib, trastuzumab (Herceptin®), gefitinib, erlotinib, methotrexate, carboplatin, docetaxel, everolimus, sorafenib, inhibitors of carbonic anhydrase, inhibitors of monocarboxylate transporters, pembrolizumab, atezolizumab, PD-1-based anticancer drugs, nivolumab, inhibitors of poly (ADP-ribose) polymerase 1 (PARP-1), inhibitors of poly (ADP-ribose) polymerase 2 (PARP-2), olaparib, rucaparib, niraparib, bevacizumab, or VEGF inhibitors, but is not limited thereto. Preferably, the anticancer agent may be a PT-based anticancer agent such as oxaliplatin, cisplatin, and carboplatin. The PT-based anticancer agent is known to induce cachexia by increasing the level of GDF-15 in a subject according to its use. When the pharmaceutical composition containing the ionic compound is used in combination with the anticancer agent or after PT-based anticancer agent treatment, it is possible to maintain activity against tumor cells as an inherent effect of an anticancer agent while suppressing weight loss and loss of appetite, which are side effects of the anticancer agent.

For example, the anti-inflammatory agent may be NSAIDs such as salicylic acid, ibuprofen, dexibuprofen, naproxen, ketoprofen, indomethacin, diclofenac, piroxicam, or meloxicam, but is not limited thereto.

For example, the antibacterial agent may be benzylpenicillin, ampicillin, streptomycin, chloramphenicol, or tetracycline, but is not limited thereto.

For example, the antifungal agent may be nystatin, amphotericin B, or trichomycin, but is not limited thereto.

For example, the antiviral agent may be oseltamivir, acyclovir, ganciclovir, sofosbuvir, or remdesivir, but is not limited thereto.

For example, the immunomodulator may be adalimumab, infliximab, etanercept or cyclosporine, but is not limited thereto.

For example, the steroid may be hydrocortisone, prednisone, triamcinolone, or fluticasone, but is not limited thereto.

Additionally, the active agents may further include anticoagulants such as warfarin and heparin; anticonvulsants such as carbamazepine and phenytoin; antidepressants such as selective serotonin reuptake inhibitors (SSRIs), serotonin-norepinephrine reuptake inhibitors (SNRIs); antioxidants such as coenzyme q10 and alpha lipoic acid; and vitamins such as vitamins A, B, C, D, and E.

The pharmaceutical composition may include one or more selected from pharmaceutically acceptable carriers, excipients, and diluents. For example, the carrier, excipient, and diluent may be lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, methylhydroxybenzoate, propylhydroxybenzoate, talc, magnesium stearate, mineral oil, etc., but are not limited thereto. The carrier, excipient, and diluent may be included within a range capable of securing an effective concentration of the ionic compound.

The pharmaceutical composition may be formulated into a liquid, powder, an agent for oral administration, an injection, an infusion, an aerosol, a tablet, a capsule, a pill, a depot, or a suppository. The pharmaceutical composition may further include a filler, an extender, a binder, a wetting agent, a disintegrant, a surfactant, an excipient, etc. as additives necessary for formulation.

The pharmaceutical composition may be administered orally, intravenously, subcutaneously, intramuscularly, or mucosally. Preferably, the pharmaceutical composition may be administered orally or intravenously. For oral administration, the pharmaceutical composition may be formulated into the above-described liquid preparation, an agent for oral administration, etc., and for intravenous administration, it may be formulated into the above-described injections, infusions, etc.

The cachexia and muscle loss may be induced by causes such as cancer, tuberculosis, diabetes, AIDS, chronic obstructive pulmonary disease, multiple sclerosis, congestive heart failure, hemophilia, hypopituitarism, or liver cirrhosis. Preferably, the cachexia and muscle loss may be caused by cancer. The cancer may be selected from the group consisting of lung cancer, breast cancer, colorectal cancer, stomach cancer, liver cancer, brain cancer, pancreatic cancer, thyroid cancer, skin cancer, bone marrow cancer, lymphoma, uterine cancer, cervical cancer, ovarian cancer, kidney cancer, and melanoma. The pharmaceutical composition provides a multimodal preventive or therapeutic effect on cachexia and muscle loss caused by the causes described above.

The muscle loss is induced by diseases such as sarcopenia, atony, muscular atrophy, muscular dystrophy, amyotrophic lateral sclerosis, or myasthenia gravis. The pharmaceutical composition provides a multimodal preventive or therapeutic effect on muscle loss caused by the above diseases.

In another embodiment of the present disclosure, there is provided a food composition containing an ionic compound, in which the ionic compound includes calcium cation, a first anion, and a second anion, wherein the first anion and the second anion are different from each other, each being independently an anion of ascorbic acid, dichloroacetic acid, branched-chain amino acid, or derivatives thereof. In the food composition, the ionic compound is replaced with the description of the previous specific embodiment.

The food composition may be a health functional food.

A health functional food may be referred to as food for special health use (FoSHU), and it means food that can be processed to efficiently display bioregulatory functions or exhibit medical efficacy in addition to supplying nutrients. For example, the health functional food may be provided in various forms such as processed foods (e.g., canned food, retort, etc.), tablets, capsules, powders, granules, liquids, and pills.

The food composition may further include commonly used food additives. For example, the food composition may include preservatives, antioxidants, coloring agents, color formers, seasonings, sweeteners, flavoring agents, expanding agents, emulsifiers, thickeners, coating agents, gum base agents, antifoaming agents, etc.

Hereinafter, the present disclosure will be described in more detail through specific Preparation Examples, Examples, and Experimental Examples. The following Preparation Examples, Examples, and Experimental Examples are intended to illustrate the present disclosure, and the present disclosure is not limited by the Preparation Examples, Examples, and Experimental Examples.

### Preparation Example 1. Preparation of Calcium Salts of Ascorbic Acid and Dichloroacetic Acid

An ascorbic acid solution was prepared by dissolving 176 mg of ascorbic acid in 125 mL of distilled water at room temperature. A dichloroacetic acid solution was prepared by dissolving 129 mg of dichloroacetic acid in 125 mL of distilled water. The ascorbic acid solution was slowly added to the dichloroacetic acid solution while stirring. After slowly adding 105 mg of calcium carbonate (CaCO₃) to the mixed solution while stirring at room temperature for 30 minutes, the reaction temperature was gradually raised to 60° C. and stirred until no more CO₂ was generated. After drying the resultant with a rotary evaporator and a vacuum oven and removing unreacted materials with diethyl ether, the resultant was subjected to filtration, drying, and pulverization to thereby obtain powdered calcium salts of ascorbic acid and dichloroacetic acid. All of the above processes were performed under a nitrogen atmosphere, and the calcium salt was named Asc-Ca-DCA or DCA-Ca-Asc.

### Preparation Example 2. Preparation of Calcium Salts of Ascorbic Acid and Leucine

A mixed solution of ascorbic acid and leucine was prepared by dissolving 1,000 mg of ascorbic acid and 745 mg of leucine in 15 mL of distilled water in a round flask at room temperature and stirring for 10 minutes. After slowly adding 568 mg of calcium carbonate (CaCO₃) to the mixed solution while stirring at room temperature for 30 minutes, the reaction temperature was gradually raised to 60° C. and stirred until no more CO₂ was generated. After drying the resultant with a rotary evaporator and a vacuum oven and removing unreacted materials with diethyl ether, the resultant was subjected to filtration, drying, and pulverization to thereby obtain powdered calcium salts of ascorbic acid and leucine. All of the above processes were performed under a nitrogen atmosphere, and the calcium salt was named Asc-Ca-Leu or Leu-Ca-Asc.

### Preparation Example 3. Preparation of Calcium Salts of Dichloroacetic Acid and Leucine

A mixed solution of ascorbic acid and leucine was prepared by dissolving 1,000 mg of dichloroacetic acid and 1,018 mg of leucine in 15 mL of distilled water in a round flask at room temperature and stirring for 10 minutes. After slowly adding 777 mg of calcium carbonate (CaCO₃) to the mixed solution while stirring at room temperature for 30 minutes, the reaction temperature was gradually raised to 60° C. and stirred until no more CO₂ was generated. After drying the resultant with a rotary evaporator and a vacuum oven and removing unreacted materials with diethyl ether, the resultant was subjected to filtration, drying, and pulverization to thereby obtain powdered calcium salts of dichloroacetic acid and leucine. All of the above processes were performed under a nitrogen atmosphere, and the calcium salt was named DCA-Ca-Leu or Leu-Ca-DCA.

### Experimental Examples 1-1 and 1-2. Confirmation of Cytotoxicity of Ionic Compounds

### Experimental Example 1-1. Confirmation of Cytotoxicity to Cells (Myoblast Cells) Before C2C12 Differentiation

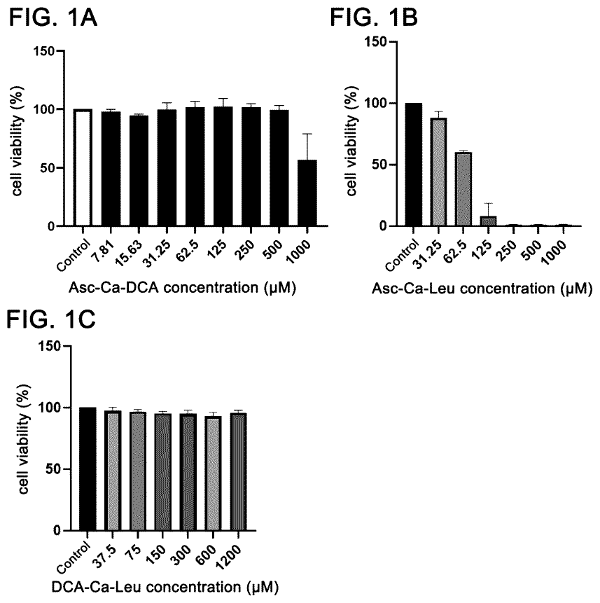

A C2C12 cell line was dispensed into a 96-well plate at 1.0×10⁴ cells/well, and then cultured in DMEM medium containing 10% fetal bovine serum (FBS) under 37° C. and 5% CO₂ conditions for one day. When the cell density reached about 70%, each of the calcium salts of Preparation Examples 1 to 3 were added to each well at 8 predetermined concentrations, respectively, and diluted by 1/2 to confirm cytotoxicity. After culturing for 72 hours, the samples were treated with the MTT solution and cultured in a dark incubator for 3 hours. After removing the supernatant, 100 μL of DMSO was added to each well, and the optical density (OD) was measured at 560 nm to confirm cell toxicity. The experiment was repeated three times, and cell viability results are shown in FIG. 1A to 1C.

Referring to FIG. 1A, the calcium salt of Preparation Example 1 exhibited toxicity with a cell viability decrease of approximately 50% at the treatment concentration of 1,000 μM. Referring to FIG. 1B, the calcium salt of Preparation Example 2 exhibited toxicity with a cell viability decrease of approximately 50% at the treatment concentration of 100 μM. Referring to FIG. 1C, the calcium salt of Preparation Example 3 exhibited no toxicity up to treatment concentrations of 1,200 μM.

### Experimental Example 1-2. Confirmation of Cytotoxicity to Cells (Myotube Cells) after C2C12 Differentiation

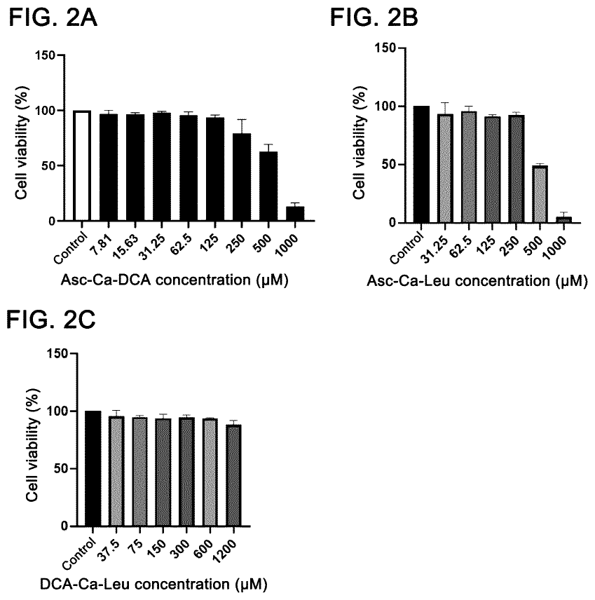

A C2C12 cell line was dispensed into a 96-well plate at 1.5×10⁴ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days, and each of the calcium salts of Preparation Examples 1 to 3 was added to each well at 8 predetermined concentrations, respectively, and diluted by 1/2 to confirm cytotoxicity. After culturing for 72 hours, the samples were treated with the MTT solution and cultured in a dark incubator for 3 hours. After removing the supernatant, 100 μL of DMSO was added to each well, and the optical density (OD) was measured at 560 nm to confirm cell toxicity. The experiment was repeated three times, and cell viability results are shown in FIGS. 2A to 2C.

Referring to FIG. 2A, the calcium salt of Preparation Example 1 exhibited toxicity with a cell viability decrease of approximately 50% at the treatment concentration of 500 μM. Referring to FIG. 2B, the calcium salt of Preparation Example 2 exhibited toxicity with a cell viability decrease of approximately 50% at the treatment concentration of 500 μM. Referring to FIG. 2C, the calcium salt of Preparation Example 3 exhibited no toxicity up to treatment concentrations of 1,200 μM.

### Experimental Examples 2-1 to 2-3. Preparation of Cellular Model in Cachexia State

### Experimental Example 2-1. Confirmation of Differentiation Conditions of Mouse Myoblast Cell Line (C2C12)

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the cells were washed with DPBS and the medium was replaced with a differentiation medium (DMEM) containing 2% horse serum and the cells were cultured for 3 days.

### Experimental Example 2-2. Preparation of Cancer-Cell Conditioned Medium (CCM)

Mouse colorectal cancer cell line CT26 was dispensed into a 100-mm culture plate at 2.0×10⁶ cells/well and cultured in DMEM medium containing 10% fetal bovine serum (FBS) under 37° C. and 5% CO₂ conditions for 48 hours. Thereafter, the medium was replaced with a serum-free medium and the medium was collected after 24 hours. The collected medium was centrifuged at 1,500 rpm for 3 minutes to remove cell debris, and the final collection was obtained using a 0.22-μm filter.

### Experimental Example 2-3. Preparation of Cellular Model in Cachexia State

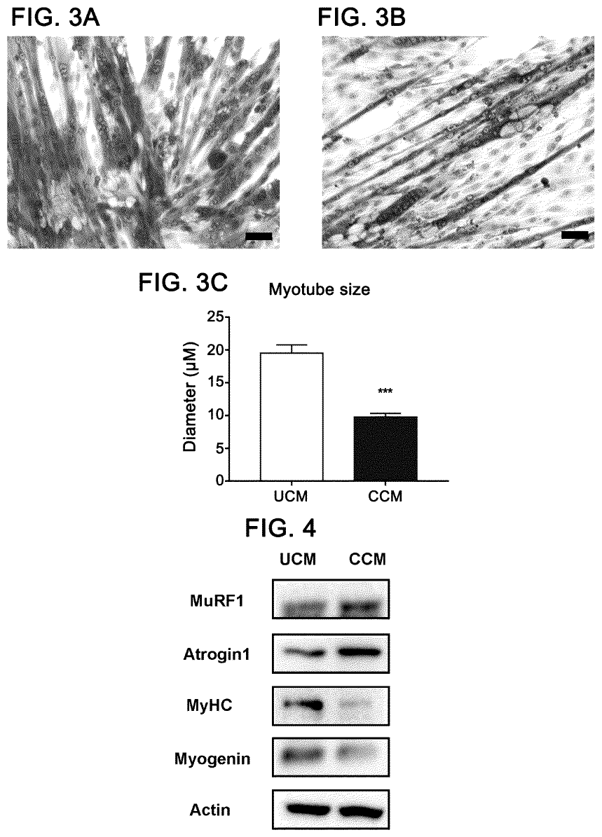

C2C12, which was differentiated into myotube cells by replacing with a differentiation medium, was treated with a mixture in which a differentiation medium and a prepared cancer cell culture medium (CCM) were mixed at a ratio of 1:1, and the cells were cultured for 72 hours. In order to compare the size of the myotube diameter with that of the normal control group, staining was performed using Crystal violet staining, and each image was confirmed and the results are shown in FIG. 3. FIG. 3A shows the image of a normal control (unconditioned medium; UCM), and FIG. 3B shows the image of CCM. The scale bars inserted in FIG. 3A and FIG. 3B represent a horizontal length of 50 μm. After the staining, the myotube diameter was measured using Image J and the results are shown in FIG. 3C. Referring to FIG. 3, the decrease of myotube cells in CCM was confirmed.

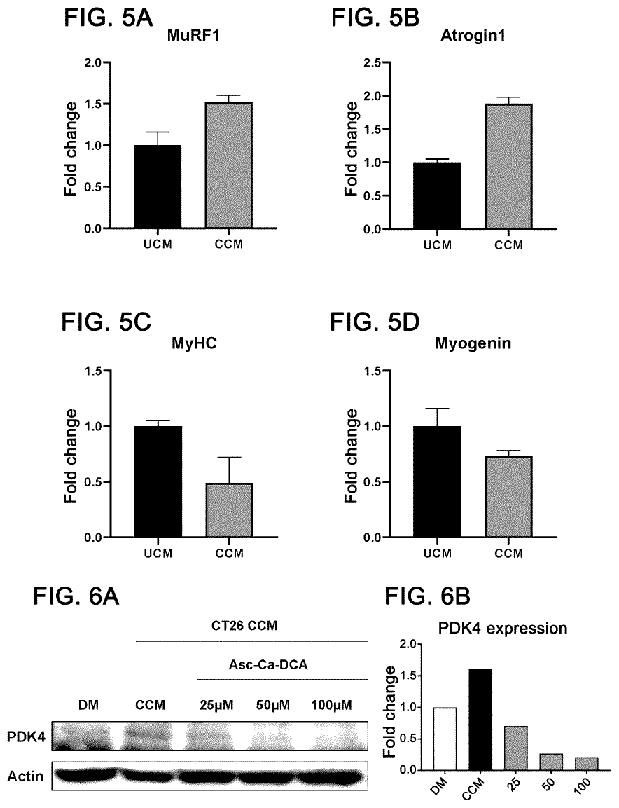

Subsequently, MuRF1, Atrogin1, MyHC, and Myogenin, which are markers relating to degradation and differentiation of muscle in the treatment group (CCM) and the normal control group (UCM), were analyzed using Western blot. The results are shown in FIG. 4, and the relative expression level (fold change; FC) for each marker is shown in FIGS. 5A to 5D.

Referring to FIG. 3C, the myotube diameter in the treated group (CCM) was reduced to about 50% of that in the normal control group (UCM). Referring to FIGS. 5A to 5D, it was confirmed that while the expression of muscle degradation markers (MuRF1 and Atrogin1) was increased, the expression of muscle differentiation markers (MyHC and Myogenin) was decreased.

### Experimental Examples 3-1 and 3-2. Confirmation of Expression of Target Markers According to Treatment with Calcium Salt of Preparation Example 1

### Experimental Example 3-1. Confirmation of Changes in Expression Level of PDK-4

C2C12 cell line was dispensed into a 6-well plate at 4.0×10⁵ cells/well and cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming the completion of differentiation of the cells, the cells were treated with a mixture, in which the differentiation medium and cancer-cell conditioned medium (CCM) were mixed at a 1:1 ratio, to form a control group, and simultaneously, a test group was set up by adding the calcium salt (Asc-Ca-DCA) of Preparation Example 1 at three predetermined concentrations. After culturing for 72 hours, the cells were collected and analyzed for PDK-4, which is a target marker, by Western blot and the results are summarized in FIG. 6.

FIG. 6A shows a Western blot result and FIG. 6B shows a graph summarizing the PDK-4 expression level from the Western blot result. Referring to FIG. 6, it was confirmed that the control group (CCM) treated with only cancer-cell conditioned medium showed an increase in the expression level of PDK-4, but the expression was inhibited by the treatment of Asc-Ca-DCA.

### Experimental Example 3-2. Confirmation of Changes in Expression Level of Calpain 3

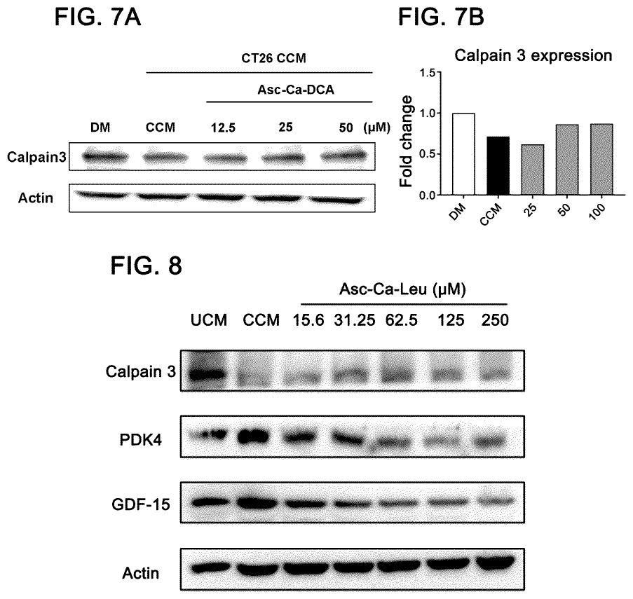

The experiment was performed in the same manner as in Experimental Example 3-1, except that Calpain 3 was analyzed as a target marker for Western blot, and the results are summarized in FIG. 7.

FIG. 7A shows a Western blot result and FIG. 7B shows a graph summarizing the expression level of Calpain 3 from the Western blot result. Referring to FIG. 7, it was confirmed that the control group (CCM) treated with only cancer-cell conditioned medium showed a decrease in the expression level of Calpain 3, but it was confirmed that the decrease in expression was inhibited by the treatment of Asc-Ca-DCA.

### Experimental Examples 4-1 to 4-3. Confirmation of Expression of Target Markers According to Treatment with Calcium Salt of Preparation Example 2

### Experimental Example 4-1. Confirmation of Changes in Expression Level of Calpain 3

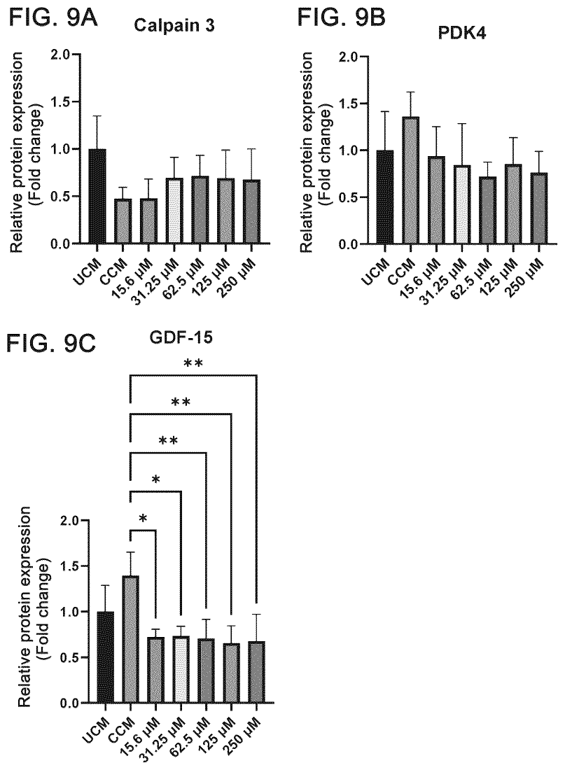

The target marker, Calpain 3, was analyzed in the same manner as in Experimental Example 3-1 except for using the calcium salt of Preparation Example 2, and the results are summarized in FIGS. 8 and 9.

### Experimental Example 4-2. Confirmation of Changes in Expression Level of PDK-4

The analysis was performed in the same manner as in Experimental Example 4-1 except for analyzing PDK-4 as a target marker for a Western blot, and the results are summarized in FIGS. 8 and 9.

### Experimental Example 4-3. Confirmation of Changes in Expression Level of GDF-15

The analysis was performed in the same manner as in Experimental Example 4-1 except for analyzing GDF-15 as a target marker for a Western blot, and the results are summarized in FIGS. 8 and 9.

FIG. 8 shows a Western blot result and FIG. 9 shows a graph summarizing the expression level of Calpain 3 (FIG. 9A), the expression level of PDK-4 (FIG. 9B), and the expression level of GDF-15 (FIG. 9C) from the western blot result. Referring to FIG. 9A, it was confirmed that the control group (CCM) treated with only cancer-cell conditioned medium showed a decrease in the expression level of Calpain 3, but it was confirmed that the decrease in expression was inhibited by the treatment of Asc-Ca-Leu. Referring to FIGS. 9B and 9C, the control group (CCM) treated only with cancer-cell conditioned medium showed an increase in the expression levels of PDK-4 and GDF-15, but the expression was inhibited by the treatment of Asc-Ca-Leu.

### Experimental Examples 5-1 to 5-3. Confirmation of Expression of Target Markers According to Treatment with Calcium Salt of Preparation Example 3

### Experimental Example 5-1. Confirmation of Changes in Expression Level of Calpain 3

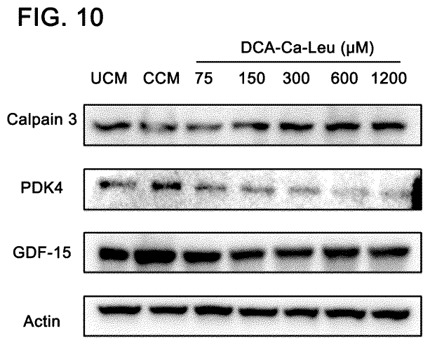

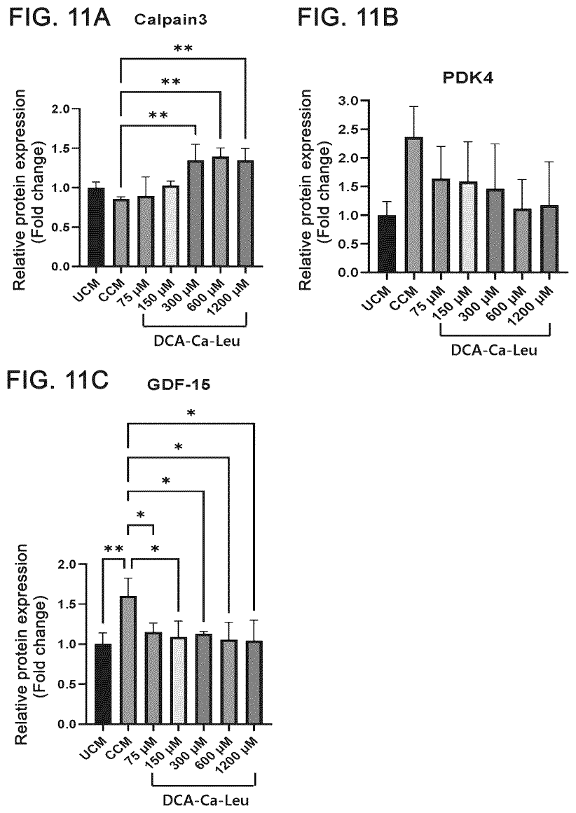

The target marker, Calpain 3, was analyzed in the same manner as in Experimental Example 3-1 except for using the calcium salt of Preparation Example 3, and the results are summarized in FIGS. 10 and 11.

### Experimental Example 5-2. Confirmation of Changes in Expression Level of PDK-4

The analysis was performed in the same manner as in Experimental Example 3-1 except for analyzing PDK-4 as a target marker for Western blot, and the results are summarized in FIGS. 10 and 11.

### Experimental Example 5-3. Confirmation of Changes in Expression Level of GDF-15

The analysis was performed in the same manner as in Experimental Example 3-1 except for analyzing PDK-4 as a target marker for Western blot, and the results are summarized in FIGS. 10 and 11.

FIG. 10 shows a Western blot result and FIG. 11 shows a graph summarizing the expression level of Calpain 3 (FIG. 11A), the expression level of PDK-4 (FIG. 11B), and the expression level of GDF-15 (FIG. 11C) from the western blot result. Referring to FIG. 11A, it was confirmed that the control group (CCM) treated only with cancer-cell conditioned medium showed a decrease in the expression level of Calpain 3, but it was confirmed that the decrease in expression was inhibited by the treatment with DCA-Ca-Leu. Referring to FIGS. 11B and 11C, the control group (CCM) treated only with cancer-cell conditioned medium showed an increase in the expression levels of PDK-4 and GDF-15, but the expression was inhibited by the treatment of DCA-Ca-Leu.

### Experimental Examples 6-1 and 6-2. Confirmation of Correlation with Target Markers and Effect of Inducing Cachexia

### Experimental Example 6-1. Confirmation of Changes in Target Markers According to Treatment of Cancer-Cell Conditioned Medium (CCM) in C2C12 Myotube Cells

PDK-4, the target gene of Forkhead box protein O (FOXO), which is closely related to energy metabolism among the exacerbation mechanisms of cancer-derived cachexia and is the core of the mechanism relating to protein degradation in muscle cells, is known to be overexpressed when the subject exhibits cachexia symptoms. It was confirmed whether the expression of PDK-4 is increased compared to that of the normal control group, when myotube cells were treated with cancer-cell conditioned medium (CCM) to create a cancer cachexia situation.

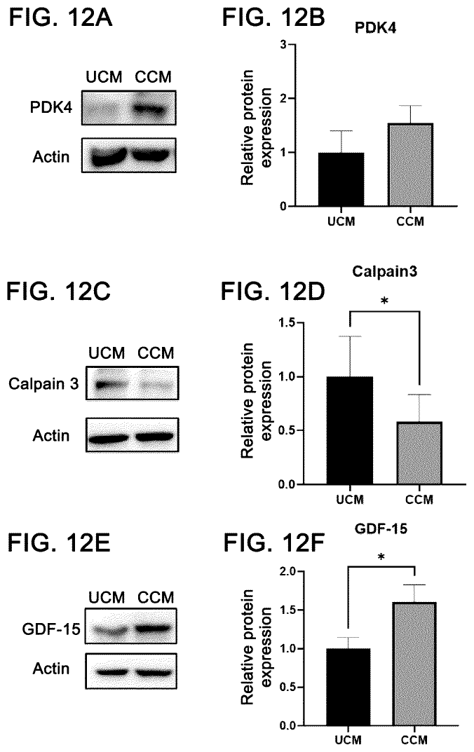

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation of the cells, the cells were treated with a mixture, in which the differentiation medium and cancer-cell conditioned medium (CCM) were mixed at a 1:1 ratio, and the cells were cultured for 72 hours, and then the cells were harvested and analyzed by Western blot. The results are summarized in FIGS. 12A and 12B.

In the same manner, Calpain 3, which is known to be underexpressed in muscle in cancer cachexia, was also analyzed using Western blot and the results are summarized in FIGS. 12C and 12D. In the same manner, GDF-15, which is known to be overexpressed in muscle in cancer cachexia, was also analyzed by Western blot and the results are summarized in FIGS. 12E and 12F.

### Experimental Example 6-2. Confirmation of Induction of Cancer Cachexia According to Changes in Expression of Target Markers

PDK-4 and Calpain 3, which are target markers, are each known to be overexpressed and underexpressed in the cancer cachexia state. It was confirmed whether the cancer cachexia state could be induced in the situation where the expression level of PDK-4 has been increased, and whether changes in muscle degradation and differentiation markers were the same as in the cachexia state.

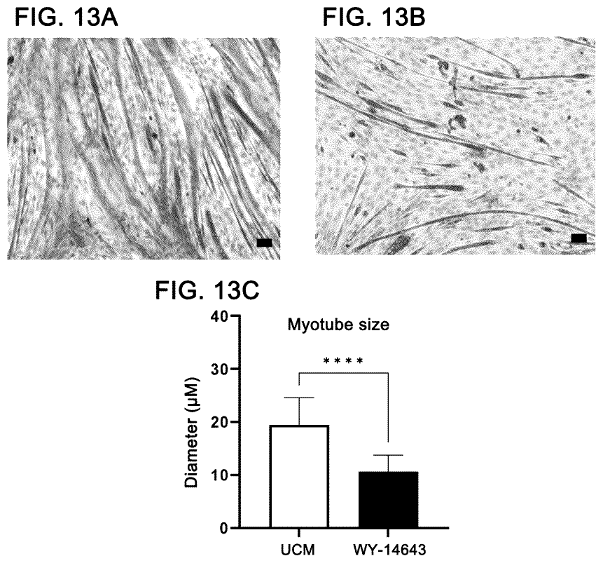

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days to set up a normal control group, and simultaneously a test group was set up by treatment with WY-14643, which is a PDK-4 activator, at a concentration of 50 μM. After culturing for 72 hours, the cells were stained with Crystal violet staining, and each of the images is shown in FIG. 13. FIG. 13A shows an image of a normal control group (UCM), and 13B shows an image treated with an activator (WY-14643). Scale bars inserted in FIGS. 13A and 13B represent a horizontal length of 50 μm. After the staining, the myotube diameter was measured using Image J and the results are shown in FIG. 13C. Referring to FIG. 13, it was confirmed that the diameter of myotube cells decreased when treated with the PDK-4 activator (i.e., WY-14643), thereby inducing the cells to a state similar to that of cancer cachexia.

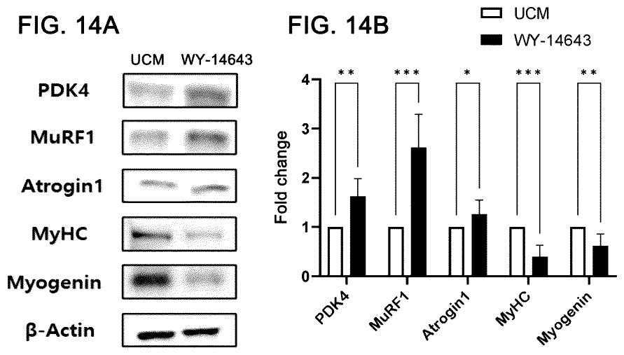

Subsequently, the expression of PDK-4, MuRF1, Atrogin1, MyHC, and Myogenin in the normal control group (UCM) and the activator-treated group (WY-14643) were analyzed by Western blot, and the results are shown in FIGS. 14A and 14B. Referring to FIG. 14, PDK-4, MuRF1, and Atrogin1 were up-regulated in the group treated with WY-14643 (i.e., an activator), and MyHC and Myogenin were down-regulated, thus confirming that a state similar to cancer cachexia was induced.

### Experimental Example 6-3. Confirmation of Induction of Cancer Cachexia According to Changes in Expression of Target Markers

It was confirmed whether the state of cancer cachexia was induced in the situation in which the expression level of Calpain 3 was reduced, and it was confirmed whether changes in markers for muscle degradation and differentiation were the same as in the state of cachexia.

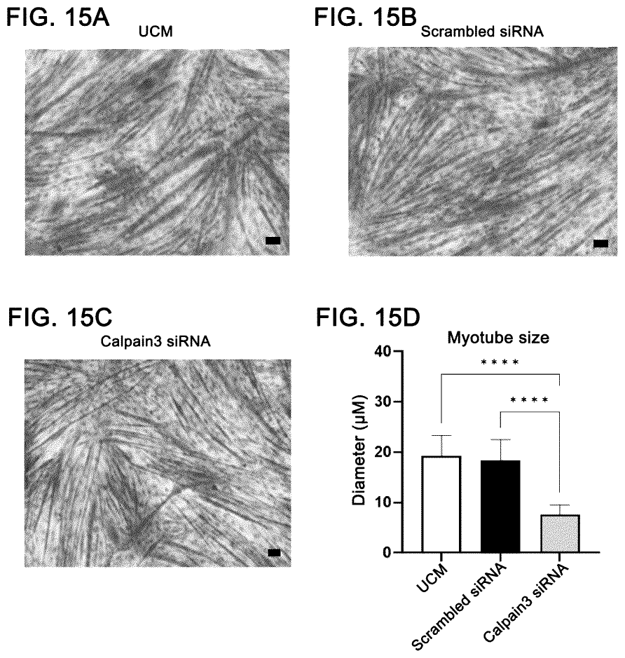

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days to set up a normal control group, and simultaneously transfected by treating with Calpain 3 siRNA and vehicle (using saline as an excipient) control scrambled siRNA at 100 nM each. The transfection was performed using Lipofectamine™ 3000 (Thermo Fisher Scientific) in accordance with the manufacturer's protocol. After culturing for 72 hours, the cells were stained with Crystal violet staining, and each image is shown in FIG. 15. FIG. 15A shows an image of the normal control group (UCM), FIG. 15B shows an image of the vehicle control group (scrambled siRNA), and FIG. 15C shows an image of the Calpain 3 siRNA treated group. Scale bars inserted in A to C of FIG. 15 represent a horizontal length of 50 μm. After the staining, the myotube diameter was measured using Image J and the results are shown in FIG. 15D. Referring to FIG. 15, under the Calpain 3 knockdown condition by Calpain 3 siRNA transfection, the myotube diameter was decreased with statistical significance compared to that of the control group, thus confirming that a state similar to cancer cachexia was induced.

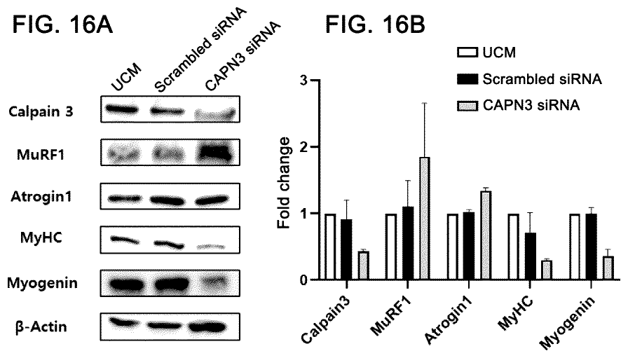

Subsequently, the expression of Calpain 3, MuRF1, Atrogin1, MyHC, and Myogenin in the normal control group (UCM), vehicle control group (scrambled siRNA), and Calpain 3 siRNA treated group was analyzed by Western blot, and the results are shown in FIGS. 16A and 16B. Referring to FIG. 16, it was confirmed that the expression level of Calpain 3 was decreased according to Calpain 3 siRNA transfection, whereas MuRF1 and Atrogin1 were up-regulated, and MyHC and Myogenin were down-regulated, thus confirming of induction into a state similar to cancer cachexia.

### Experimental Example 7. Confirmation of Effect of Calcium Salt of Preparation Example 1 on Inhibition of COX2

The calcium salt of Preparation Example 1 was mixed with DMSO, and the mixture was added to 200 mM Tris-HCl buffer (pH 8.0, containing 6 μM EDTA and 10 μM hematin) (in which the DMSO concentration is 1% in the final buffer solution). At this time, the concentration of the calcium salt contained in the buffer solution was set to 10 μM. As an enzyme, 34 U/mL of human recombinant cyclooxygenase COX-2 expressed in insect Sf21 cells was further added to the buffer solution, and pre-reacted at 25° C. for 15 minutes. In addition, 3 μM arachidonic acid and 100 μM Ampliflu Red were added and reacted at 25° C. for 3 minutes. The amount of Resofurin produced in the reaction process was measured at 535 nm/590 nm by a spectrofluorescence method, and the inhibitory effect on COX2 was confirmed by comparing with the calcium salt untreated group.

Calcium salt (Asc-Ca-DCA) of Preparation Example 1 was confirmed to have a 74% inhibitory effect on COX2; therefore, it was determined as being able to provide preventive or therapeutic effects on cachexia and muscle loss.

### Experimental Examples 8-1 and 8-2. Confirmation of Inhibitory Effect of Calcium Salts of Preparation Examples on Decrease of C2C12 Myotube Differentiation

### Experimental Example 8-1. Measurement of Myotube Cell Diameter of C2C12

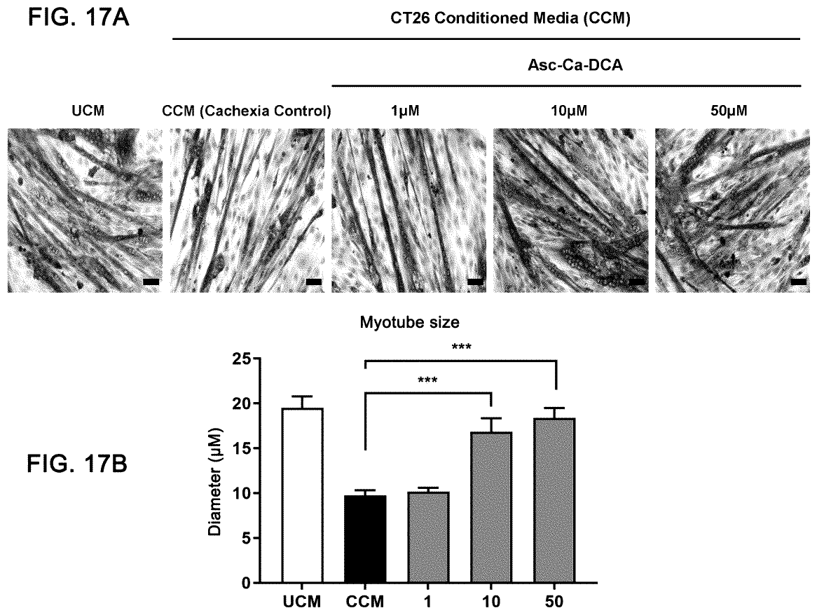

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation of the cells, the cells were treated with a mixture, in which the differentiation medium and cancer-cell conditioned medium (CCM) were mixed at a 1:1 ratio, to form a control group, and simultaneously, a test group was set up by adding the calcium salt of Preparation Example 1 at three predetermined concentrations. After culturing for 72 hours, the cells were stained with Crystal violet staining, and each image is summarized in FIG. 17A. The scale bar inserted in FIG. 17A represents a horizontal length of 50 μm. The myotube diameter was measured using Image J from the above image, and the change in myotube cell diameter is summarized in FIG. 17B.

Referring to FIG. 17, the diameter of myotubes cells treated with cancer-cell conditioned medium (CCM) decreased by approximately 50% compared to that of the normal control group (UCM), creating a condition similar to cancer cachexia. However, in the test group treated with the calcium salt (Asc-Ca-DCA) of Preparation Example 1, the diameter reduction of myotubes was significantly inhibited in a statistically from the concentration of 10 μM or more.

### Experimental Example 8-2. Confirmation of Degradation and Differentiation Markers in C2C12 Myotube Cells

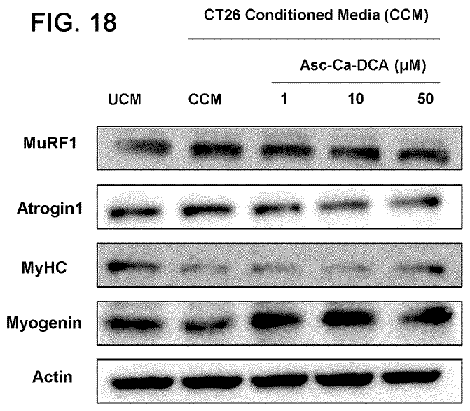

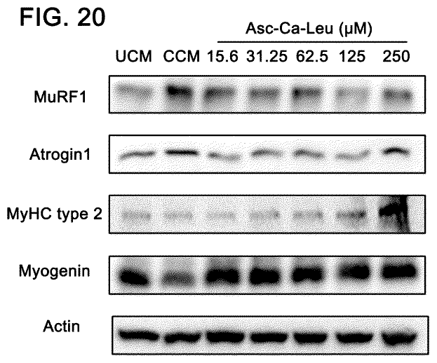

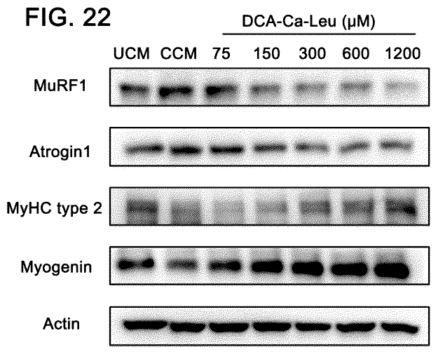

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation of the cells, the cells were treated with a mixture, in which the differentiation medium and cancer-cell conditioned medium (CCM) were mixed at a 1:1 ratio, to form a control group, and simultaneously, a test group was set up by treating with the calcium salts of Preparation Examples 1 to 3 according to three predetermined concentrations. After culturing for 72 hours, the cells were harvested and analyzed by Western blot, and the results are summarized in FIG. 18 (Preparation Example 1), FIG. 20 (Preparation Example 2), and FIG. 22 (Preparation Example 3).

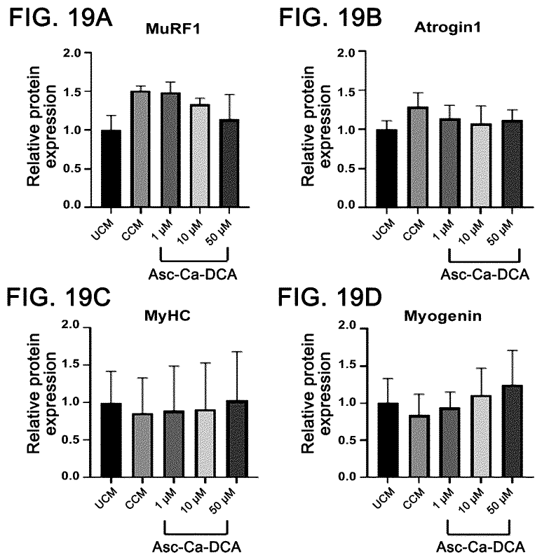

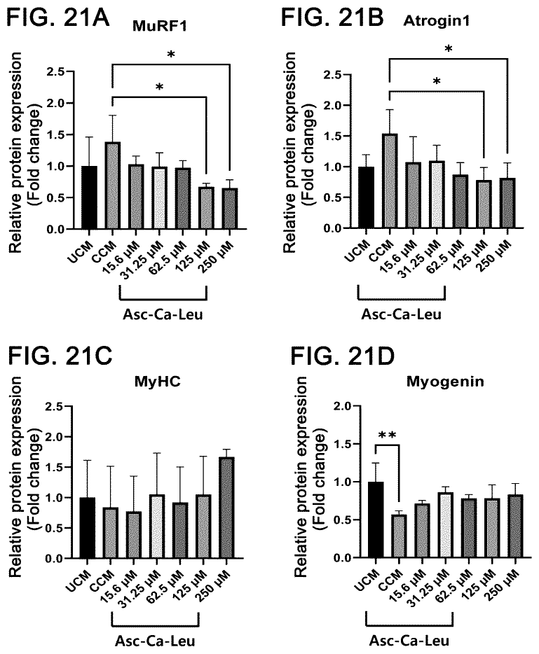

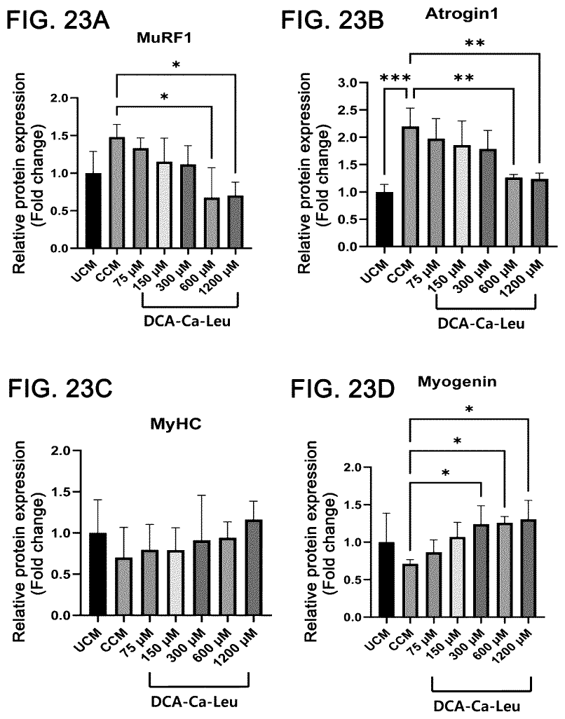

Subsequently, MuRF1, Atrogin1, MyHC, and Myogenin, which are markers relating to muscle degradation and differentiation included in each experimental group, were analyzed using Western blot, and the relative expression levels for each marker are shown in FIG. 19 (Preparation Example 1), FIG. 21 (Preparation Example 2), and FIG. 23 (Preparation Example 3).

Referring to FIGS. 18 to 23, the increase in expression of muscle degradation markers MuRF1 and Atrogin1 due to cancer-cell conditioned medium (CCM) was inhibited by treatment with calcium salts of Preparation Example 1 to 3, respectively, whereas the decrease in the expression of muscle differentiation markers MyHC and Myogenin due to cancer-cell conditioned medium (CCM) was inhibited.

### Experimental Examples 9-1 to 9-4. Comparison of Effects of Calcium Salts of Preparation Examples, Ascorbic Acid, Dichloroacetic Acid, and Combination of Ascorbic Acid and Dichloroacetic Acid

### Experimental Example 9-1. Measurement of Diameter of C2C12 Myotube Cells

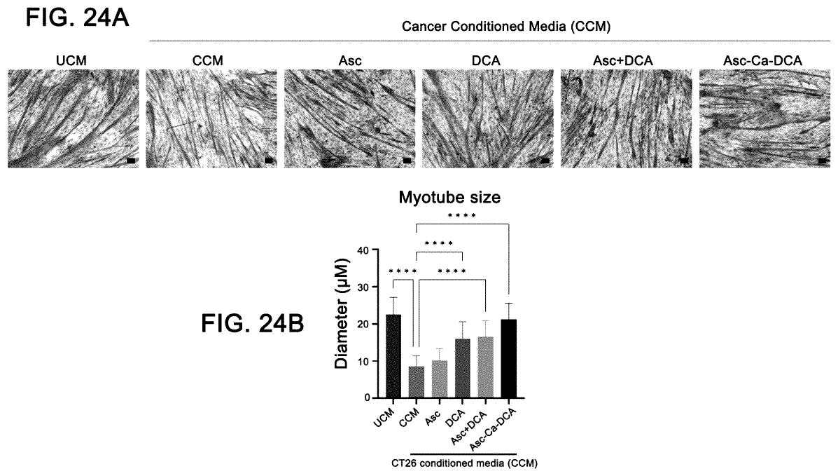

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation of the cells, the cells were treated with a mixture, in which the differentiation medium and cancer-cell conditioned medium (CCM) were mixed at a 1:1 ratio, to form a control group, and simultaneously, a test group was set up by treating with the calcium salt of Preparation Example 1 (Asc-Ca-DCA), ascorbic acid, dichloroacetic acid, and a combination of ascorbic acid and dichloroacetic acid according to predetermined concentration. After culturing for 72 hours, the cells were stained with Crystal violet staining, and the images are summarized in FIG. 24A. The scale bar inserted in FIG. 24A represents a horizontal length of 50 μm. The myotube diameter was measured using Image J from the above images, and the changes in myotube cell diameter are summarized in FIG. 24B.

Referring to FIG. 24, it was found that the diameter of myotube cells increased when the cells were treated with ascorbic acid, dichloroacetic acid, or a combination of ascorbic acid and dichloroacetic acid compared to treatment with cancer-cell conditioned medium (CCM). However, it was confirmed that treatment with the calcium salt of Preparation Example 1 increased the diameter up to a level similar to that of the normal control group (UCM).

### Experimental Example 9-2. Confirmation of Degradation/Differentiation Markers and Target Markers in C2C12 Myotube Cells

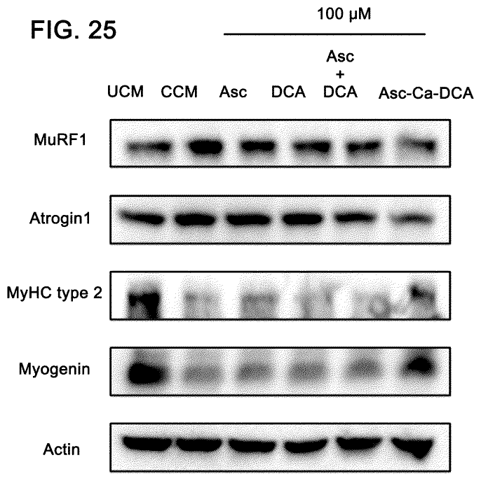

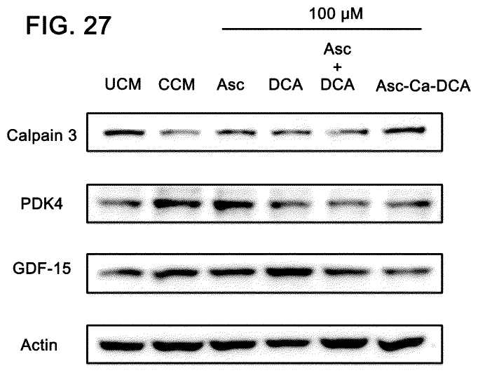

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation of the cells, the cells were treated with a mixture, in which the differentiation medium and cancer-cell conditioned medium (CCM) were mixed at a 1:1 ratio, to form a control group, and simultaneously, a test group was set up by treating with the calcium salt of Preparation Example 1 (Asc-Ca-DCA), ascorbic acid, dichloroacetic acid, and a combination of ascorbic acid and dichloroacetic acid to be suitable at predetermined concentration. After culturing for 72 hours, the cells were harvested and analyzed by Western blot, and the results are summarized in FIGS. 25 and 27.

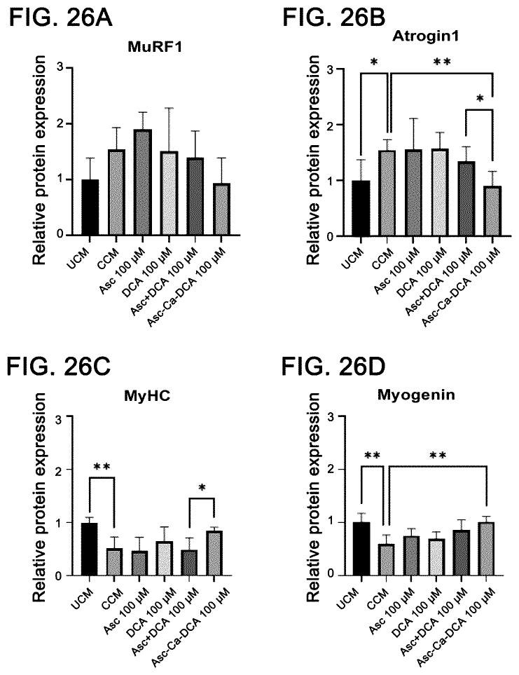

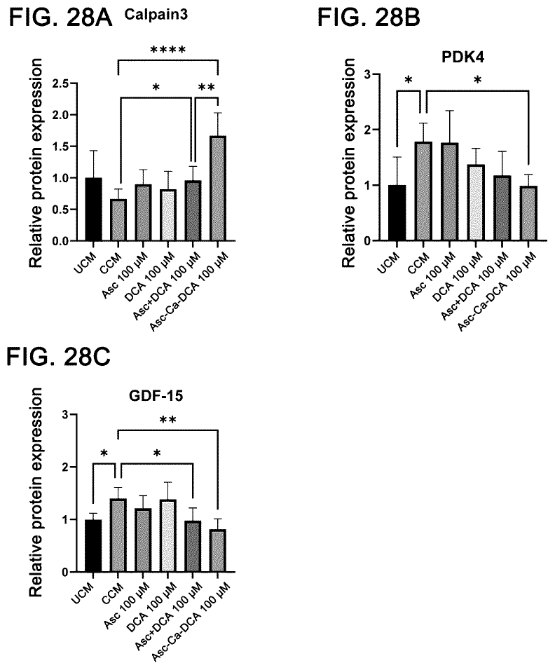

Subsequently, MuRF1, Atrogin1, MyHC, Myogenin (muscle markers) and Calpain 3, PDK-4, and GDF-15 (other target markers for muscle related disease) were analyzed using Western blot, and the relative expression levels for the markers are shown in FIGS. 26 and 28.

Referring to FIGS. 25 to 28, as for the muscle degradation markers (i.e., MuRF1 and Atrogin1), the group treated with the calcium salt (Asc-Ca-DCA) of Preparation Example 1 showed an excellent effect of inhibiting the increase of expression compared to other experimental groups, whereas as for the muscle differentiation markers (i.e., MyHC and Myogenin), the group treated with the calcium salt of Preparation Example 1 showed an excellent effect of inhibiting the decrease of expression compared to other experimental groups. The group treated with the calcium salt of Preparation Example 1 showed a higher expression level of Calpain 3 than other experimental groups, and the expressions of PDK-4 and GDF-15 were inhibited.

### Experimental Example 9-3. Confirmation of Degradation/Differentiation Markers and Target Markers in C2C12 Myotube Cells

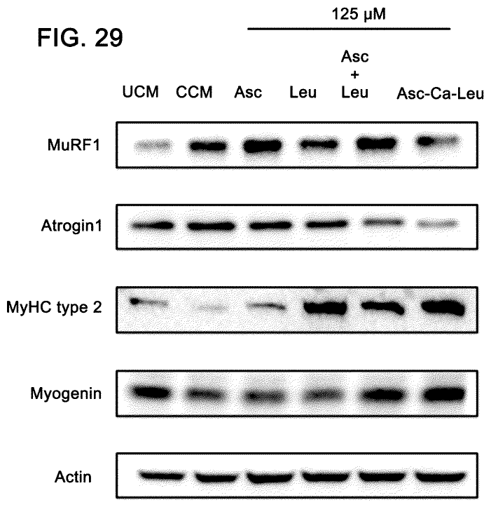

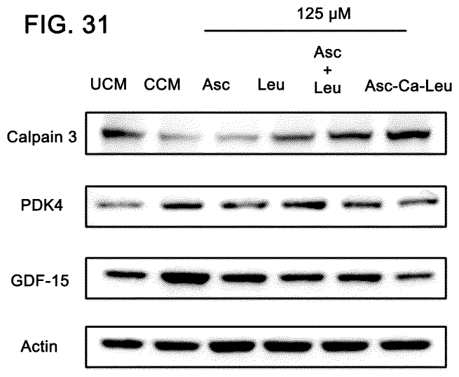

The experiment was performed in the same manner as in Experimental Example 9-2 above except for setting the experimental groups by treating with the calcium salt (Asc-Ca-Leu) of Preparation Example 2, ascorbic acid, leucine, and a combination of ascorbic acid and leucine according to the predetermined concentration. After culturing for 72 hours, the cells were harvested and analyzed by Western blot, and the results are summarized in FIGS. 29 and 31.

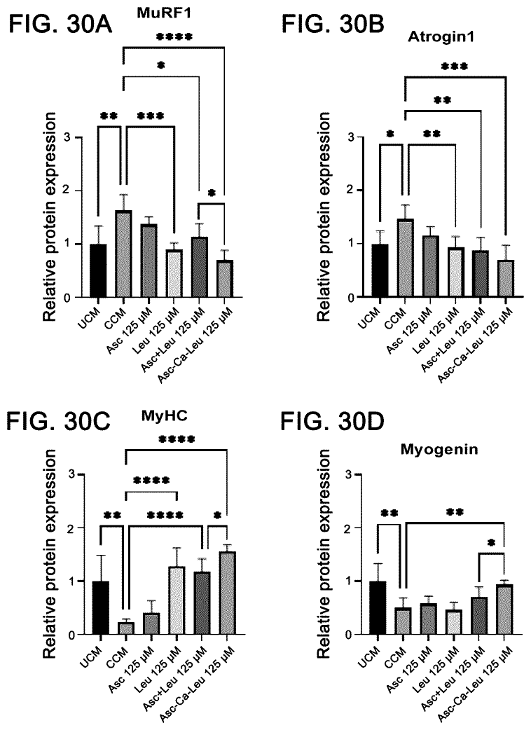

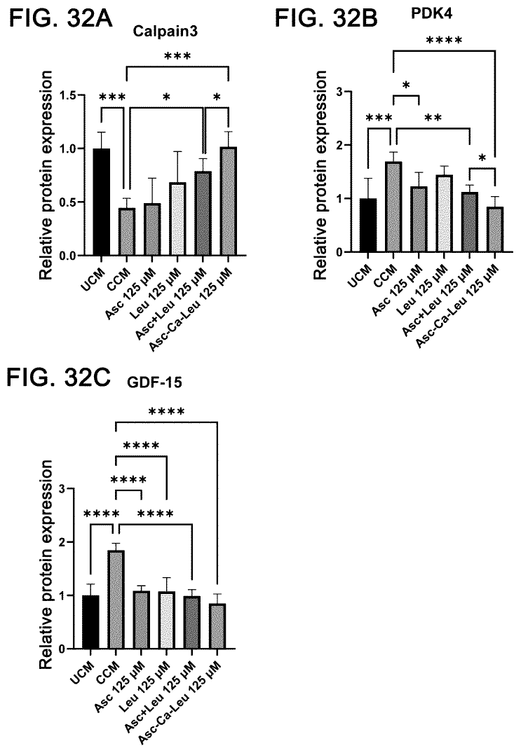

Subsequently, MuRF1, Atrogin1, MyHC, Myogenin (muscle markers) and Calpain 3, PDK-4, and GDF-15 (other target markers for muscle related disease) were analyzed using Western blot, and the relative expression levels for the markers are shown in FIGS. 30 and 32.

Referring to FIGS. 29 to 32, as for the muscle degradation markers (i.e., MuRF1 and Atrogin1), the group treated with the calcium salt (Asc-Ca-Leu) of Preparation Example 2 showed an excellent effect of inhibiting the increase of expression compared to other experimental groups, whereas as for the muscle differentiation markers (i.e., MyHC and Myogenin), the group treated with the calcium salt of Preparation Example 2 showed an excellent effect of inhibiting the decrease of expression compared to other experimental groups. The group treated with the calcium salt of Preparation Example 2 showed a higher expression level of Calpain 3 than other experimental groups, and the expressions of PDK-4 and GDF-15 were inhibited.

### Experimental Example 9-4. Confirmation of Degradation/Differentiation Markers and Target Markers in C2C12 Myotube Cells

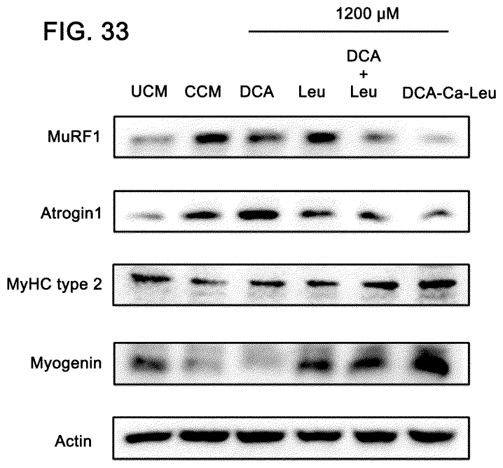

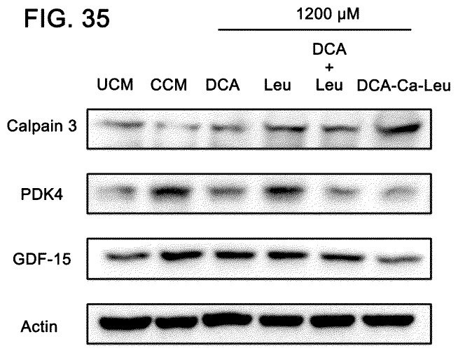

The experiment was performed in the same manner as in Experimental Example 9-2 above except for setting the experimental groups by treating with the calcium salt (DCA-Ca-Leu) of Preparation Example 3, dichloroacetic acid, leucine, and a combination of dichloroacetic acid and leucine according to the predetermined concentration. After culturing for 72 hours, the cells were harvested and analyzed by Western blot, and the results are summarized in FIGS. 33 and 35.

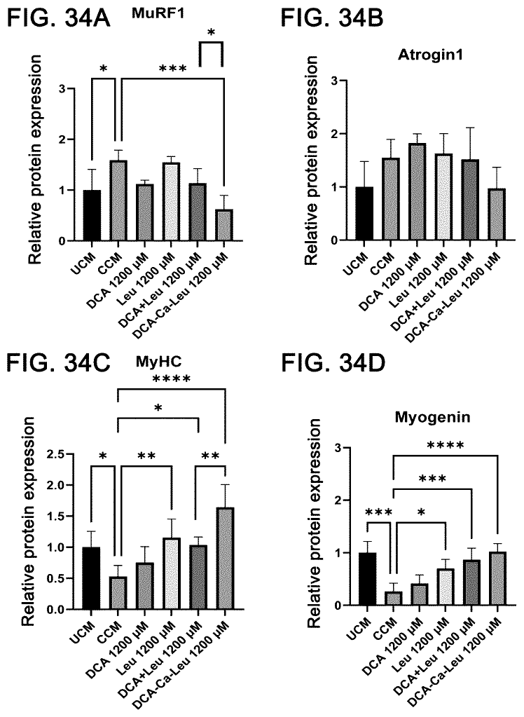

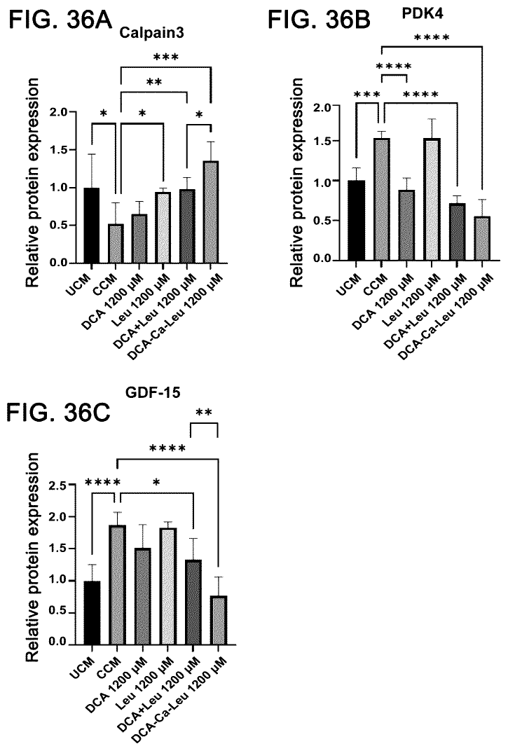

Subsequently, MuRF1, Atrogin1, MyHC, Myogenin (muscle markers) and Calpain 3, PDK-4, and GDF-15 (other target markers for muscle related disease) were analyzed using Western blot, and the relative expression levels for the markers are shown in FIGS. 34 and 36.

Referring to FIGS. 33 to 36, as for the muscle degradation markers (i.e., MuRF1 and Atrogin1), the group treated with the calcium salt (DCA-Ca-Leu) of Preparation Example 3 showed an excellent effect of inhibiting the increase of expression compared to other experimental groups, whereas as for the muscle differentiation markers (i.e., MyHC and Myogenin), the group treated with the calcium salt of Preparation Example 3 showed an excellent effect of inhibiting the decrease of expression compared to other experimental groups. The group treated with the calcium salt of Preparation Example 3 showed a higher expression level of Calpain 3 than other experimental groups, and the expressions of PDK-4 and GDF-15 were inhibited.

### Experimental Examples 10-1 to 10-3. Effect of Calcium Salts of Preparation Examples on Sarcopenia

### Experimental Example 10-1. Preparation of Sarcopenia In Vitro Model

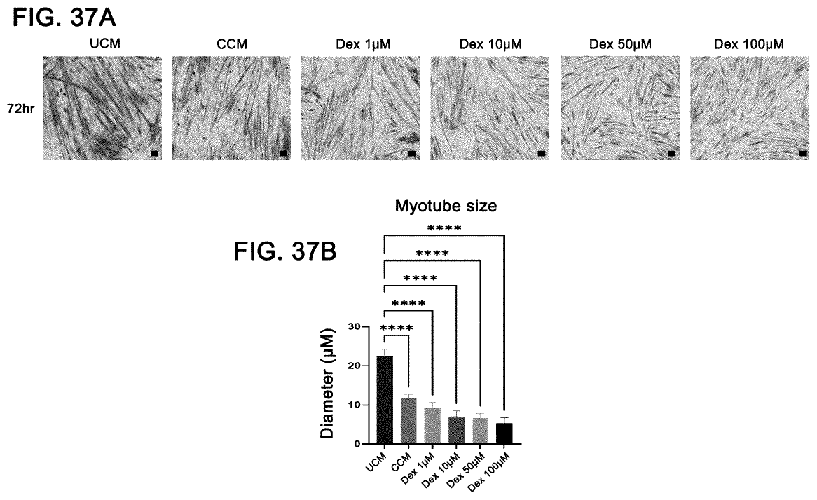

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation of the cells, experimental groups were set up by treating with dexamethasone (Dex) according to the predetermined concentrations. After culturing for 72 hours, the images stained with the Crystal violet staining method are summarized in FIG. 37A. The scale bar inserted in FIG. 37A represents a horizontal length of 50 μm. The myotube diameter was measured using Image J to confirm the changes in diameter of myotube cells, and the results are shown in FIG. 37B.

Referring to FIG. 37, it was confirmed that the myotube diameter started to decrease from the treatment concentration of 1 μM of dexamethasone. At concentrations higher than the above concentration, no statistically significant difference was confirmed from the group treated with 1 μM dexamethasone; therefore, the treatment concentration of 1 μM dexamethasone was established as an experimental condition.

### Experimental Example 10-2. Confirmation of Changes in Target Marker in Sarcopenia In Vitro Model

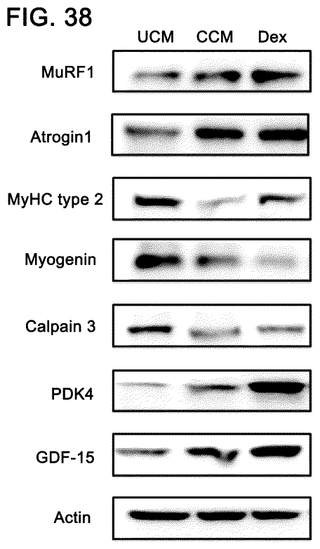

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation, the control group was treated with dexamethasone (Dex) at 1 μM, cultured for 72 hours, and the cells were harvested and subjected to Western blot for relevant markers and the results are shown in FIG. 38.

As a result of the analysis of FIG. 38, it was confirmed that the expression levels of MuRF1 and Atrogin1, which are muscle degradation markers, were increased in the dexamethasone-treated group, and the expression levels of MyHC and Myogenin, which are muscle differentiation markers, were decreased, thus confirming that the expression of relevant markers change along with muscle reduction when treated with dexamethasone. In addition, it was confirmed that the target markers are related to the main mechanism in the sarcopenia model by confirming the increase in the expression of PDK4, decrease in the expression of Calpain 3, and increase in the expression of GDF-15 in the dexamethasone-treated group.

### Experimental Example 10-3. Confirmation of Effect of Calcium Salts of Preparation Examples

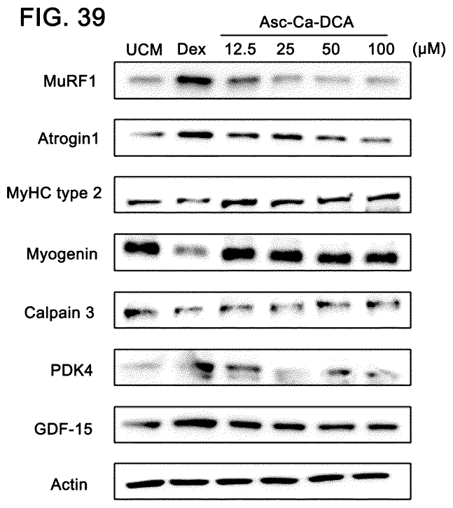

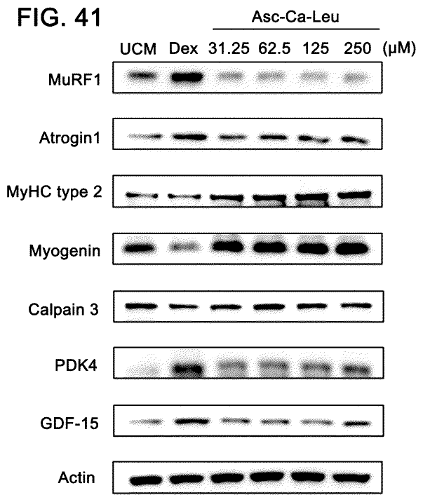

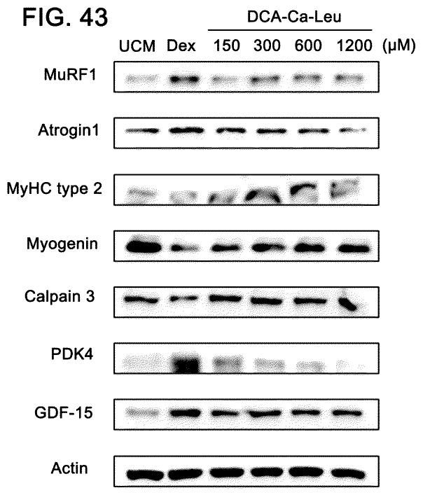

A C2C12 cell line was dispensed into a 6-well plate at 4.0×10⁵ cells/well, and then cultured in DMEM medium containing 10% fetal bovine serum (FBS) under 37° C. and 5% CO₂ conditions for one day. When the cell density reached about 80%, the medium was replaced with a differentiation medium and the cells were cultured for additional 3 days. After confirming completion of differentiation, the cells were treated with dexamethasone (Dex) at 1 μM, and experimental groups were set up by treating the calcium salts of Preparation Example 1 to 3 at predetermined concentrations. After culturing for 72 hours, the cells were harvested, and analyzed by Western blot, and the results are summarized in FIG. 39 (Preparation Example 1), FIG. 41 (Preparation Example 2), and FIG. 43 (Preparation Example 3).

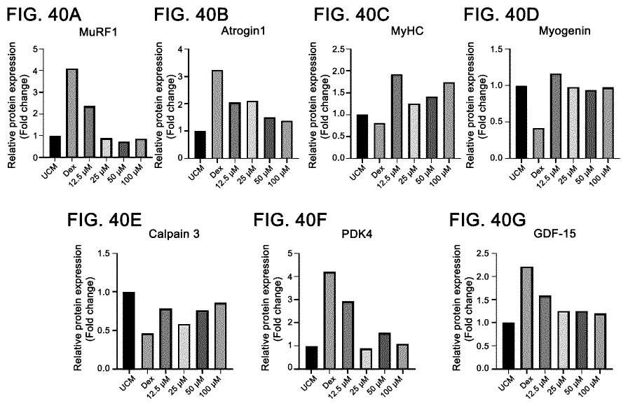

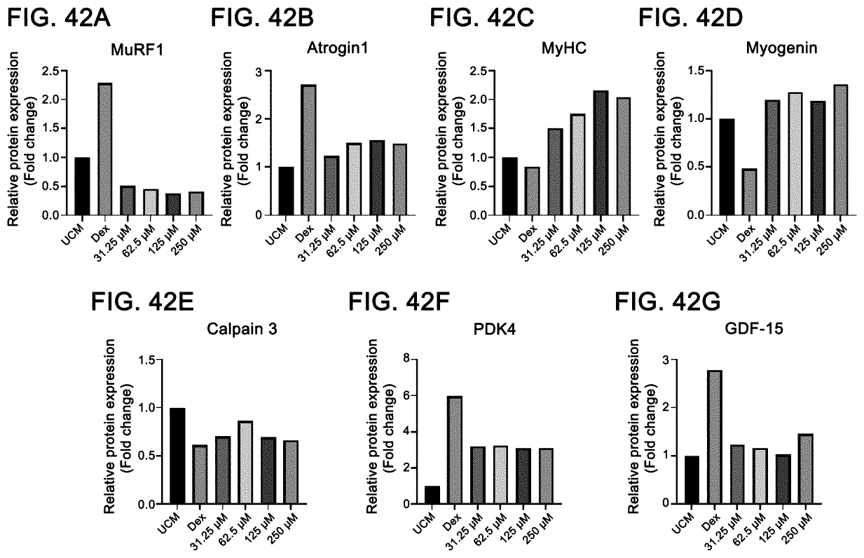

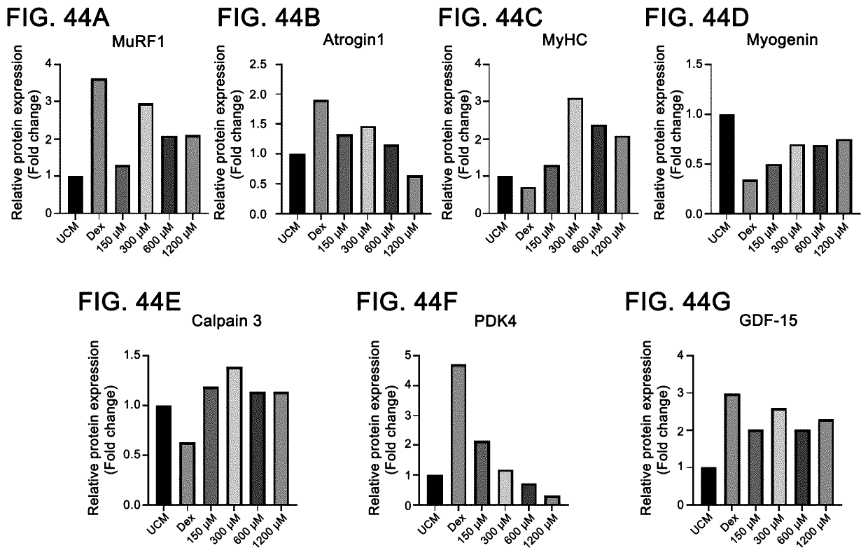

Subsequently, MuRF1, Atrogin1, MyHC, Myogenin (muscle markers), and Calpain 3, PDK-4, GDF-15 (other target markers for muscle related disease) were analyzed using Western blot, and the relative expression level of each marker is shown in FIG. 40 (Preparation Example 1), FIG. 42 (Preparation Example 2), and FIG. 44 (Preparation Example 3).

Referring to FIGS. 39 to 44, the increase of expression in MuRF1 and Atrogin1, which are muscle degradation markers, due to dexamethasone treatment was inhibited by the treatment with the calcium salts of Preparation Examples 1 to 3, respectively, whereas the decrease of expression in MyHC and Myogenin, which are muscle differentiation markers was inhibited by the treatment of dexamethasone. In addition, the decrease of expression in PDK-4, the increase of expression in Calpain 3, and the decrease of expression in GDF-15 were confirmed in the groups treated with the calcium salts of Preparation Example 1 to 3.

### Experimental Example 11. Confirmation of Drug Concentration in Blood and Pharmacokinetics Factors According to Intravenous (IV) and Oral Administration (PO) in SD Rats

The calcium salt (Asc-Ca-DCA) of Preparation Example 1 was administered intravenously (200 mg/kg, once daily) or orally (200 mg/kg, 500 mg/kg, once daily or 1,000 mg/kg twice daily). After the administration of each individual rat, blood was collected according to the elapsed time (0 to 48 hours), centrifuged, and the concentration of the administered drug in the blood and the pharmacokinetic factors (PK parameter) of the obtained plasma were analyzed through LC/MS/MS analysis, and the results are summarized in Table 1 below.

TABLE 1

| | Asc-Ca-DCA | | | | | | | |
|---|---|---|---|---|---|---|---|---|
| | | | PO | | | | | |
| | IV | | | | | | 2,000 mg/kg | |
| PK | 200 mg/kg | | 200 mg/kg | | 500 mg/kg | | (1,000 mg/kg, BID) | |
| Parameters | 1 day | 6 days | 1 day | 6 days | 1 day | 6 days | 1 day | 6 days |
| AUCₗₐₛₜ | 19.77 | 7.98 | 38.78 | 34.62 | 353.65 | 147.85 | 889.77 | 575.25 |
| (μg · h/mL) | | | | | | | | |
| Cₘₐₓ | 30.24 | 2.70 | 23.91 | 6.26 | 66.44 | 21.28 | 130.92 | 77.20 |
| (μg/mL) | | | | | | | | |
| Tₘₐₓ (h) | 0.25 | 0.00 | 0.50 | 0.00 | 1.50 | 0.00 | 1.75 | 0.00 |
| t_{1/2} (h) | 0.27 | 2.72 | 0.59 | 2.72 | 4.14 | 5.58 | 5.64 | 3.11 |

Referring to Table 1 above, in the test groups administered with the calcium salt of Preparation Example 1, the blood concentration of the calcium salt was well analyzed regardless of the administration route and concentration, and specific values for pharmacokinetic factors could be confirmed. From these, it can be determined that the calcium salt according to the Preparation Examples is maintained for a certain period of time in the body of the target subject and can provide the multimodal effect described above.

### Experimental Examples 12-1 to 12-6. In Vivo Test for Mice

### Experimental Example 12-1. Confirmation of Markers for Degradation or Differentiation in Mouse Muscle Tissue

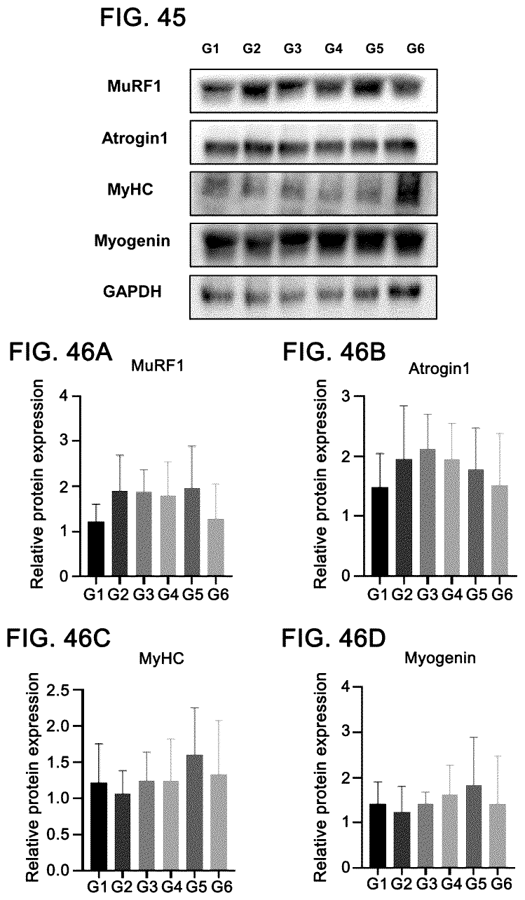

A CT26 mouse colorectal cancer cell line was subcutaneously transplanted into Balb/c mice to construct the syngeneic model, and the calcium salt (Asc-Ca-DCA) of Preparation Example 1 was administered to the mice for 21 days. The mice were sacrificed, and the skeletal muscle in the quadriceps was extracted during autopsy, and tissue was homogenized, and proteins were extracted through RIPA lysis buffer, followed by Western blot analysis. The results are summarized in FIG. 45.

Subsequently, MuRF1, Atrogin1, MyHC, and Myogenin, which are markers relating to muscle degradation and differentiation included in each experimental group, were analyzed using Western blot, and the relative expression levels of each marker are shown in FIGS. 46A to 46D.

In FIGS. 45 and 46, G1 is a normal control group without tumor, G2 is a vehicle (excipient) control group in which only vehicle (water for injection or saline) was administered to a cachexia model, G3 is a group orally administered with megestrol (125 mpk) in which megestrol was administered to a cachexia model, G4 is a group orally administered with the calcium salt of Preparation Example 1 (200 mpk) in which the calcium salt of Preparation Example 1 was administered to a cachexia model, G5 is a group intravenously administered with Preparation Example 1 (100 mpk) in which the calcium salt of Preparation Example 1 was administered to a cachexia model, and G6 is a group intravenously administered with the calcium salt of Preparation Example 1 (200 mpk) in which the calcium salt of Preparation Example 1 was administered to a cachexia model.

Referring to FIGS. 45 and 46, it was confirmed that G4 to G6, which were treated with Preparation Example 1, showed a decrease in the expression levels of muscle degradation markers (i.e., MuRF1 and Atrogin1) while showing an increase in the expression levels of muscle differentiation markers (i.e., MyHC and Myogenin) compared to the vehicle control group G2. It was confirmed that G4 to G6, which were treated with the calcium salt of Preparation Example 1, have excellent effects decreasing the expression levels of Atrogin1 and increasing the expression of Myogenin while having the effect of controlling the expression of MuRF1 and MyHC at a similar level, compared to G3 treated with megestrol, which is a therapeutic agent for cachexia.

### Experimental Example 12-2. Confirmation of Target Markers in Mouse Muscle Tissue

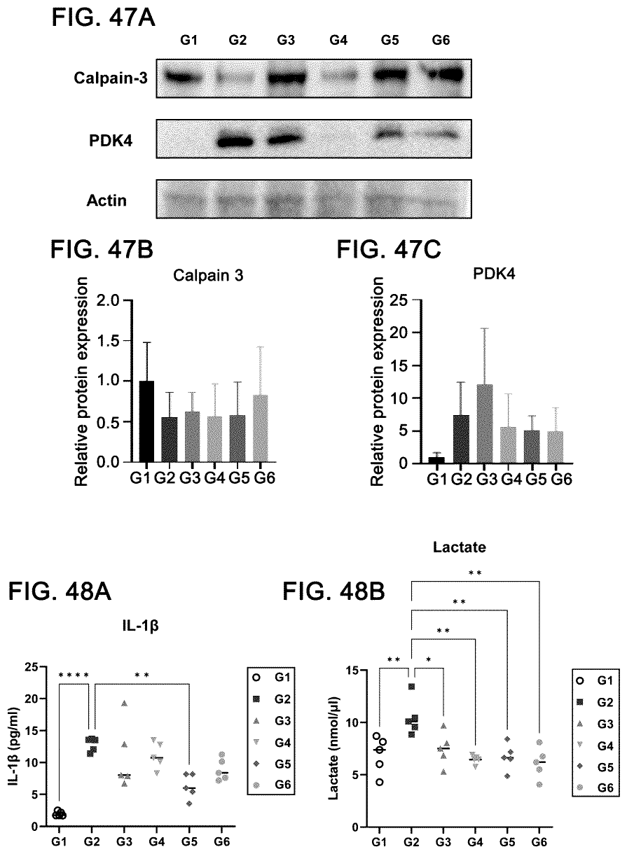

A CT26 mouse colorectal cancer cell line was subcutaneously transplanted into Balb/c mice to construct a syngeneic model, and the calcium salt (Asc-Ca-DCA) of Preparation Example 1 was administered to the mice for a total of 21 days. The mice were sacrificed, and the skeletal muscle in the quadriceps was extracted during autopsy, and tissue was homogenized, and proteins were extracted through RIPA lysis buffer, followed by Western blot analysis. The results are summarized in FIG. 47A.

Subsequently, Calpain 3 and PDK-4 included in each experimental group were analyzed using Western blot, and the relative expression levels of each marker are shown in FIGS. 47B and 47C.

In FIG. 47, each group represents the following. G1: normal control group, G2: the a vehicle (excipient) administration group, G3: megestrol administration group (125 mpk), G4: calcium salt of Preparation Example 1 oral administration group (200 mpk), G5: the calcium salt of Preparation Example 1 (100 mpk) intravenous administration group, and G6: the calcium salt of Preparation Example 1 (200 mpk) intravenous administration group.

Referring to FIG. 47, it was confirmed that G4 to G6 treated with the calcium salt of Preparation Example 1 showed an increase in the expression level of Calpain 3 while showing a decrease in the expression level of PDK-4, compared to the vehicle control group G2. It was confirmed that G6 intravenously administered with the calcium salt of Preparation Example 1 showed an excellent effect of increasing the expression level of Calpain 3, whereas G4 to G6 showed an excellent effect of reducing the expression level of PDK-4, compared to G3 treated with megestrol, which is a therapeutic agent for cachexia.

### Experimental Example 12-3. Measurement of Cytokines and Lactate in Mouse Blood

A CT26 mouse colorectal cancer cell line was subcutaneously transplanted into Balb/c mice to construct a syngeneic model, and the calcium salt (Asc-Ca-DCA) of Preparation Example 1 was administered to the mice for a total of 21 days. The mice were sacrificed, and IL-1β and lactate were analyzed using the blood serum collected at autopsy, and the results are summarized in FIGS. 48A and 48B.

In FIG. 48, each group represents the following. G1: normal control group, G2: the a vehicle (excipient) administration group, G3: megestrol administration group (125 mpk), G4: calcium salt of Preparation Example 1 oral administration group (200 mpk), G5: the calcium salt of Preparation Example 1 (100 mpk) intravenous administration group, and G6: the calcium salt of Preparation Example 1 (200 mpk) intravenous administration group.

Referring to FIG. 48, it was confirmed that G4 to G6 treated with the calcium salt of Preparation Example 1 showed a decrease in the expression levels of IL-1ß and lactate, compared to a vehicle control group G2. G4 to G6 showed the effect of reducing IL-1ß and lactate at a similar reducing effect compared to G3 treated with megestrol, which is a therapeutic agent for cachexia.

### Experimental Example 12-4. Confirmation of Effect of Inhibiting Weight Loss by Treatment with Calcium Salt of Preparation Example 1 in Cancer Cachexia Animal Model

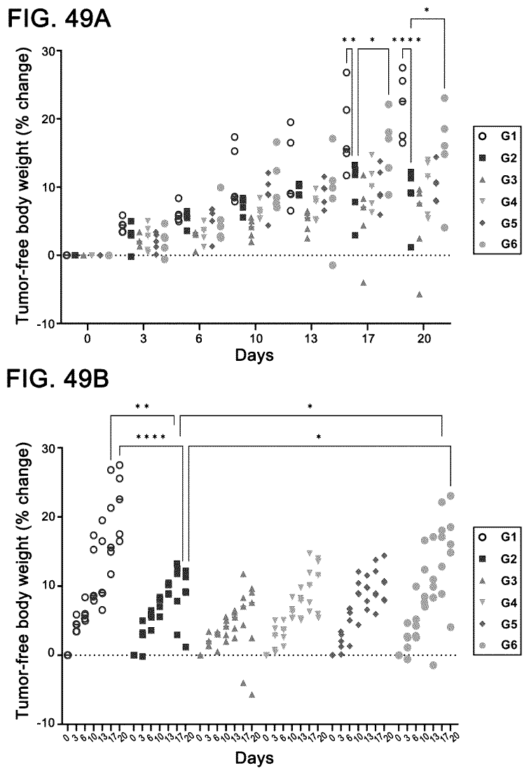

A CT26 mouse colorectal cancer cell line was subcutaneously transplanted into Balb/c mice to construct a syngeneic model, and the calcium salt (Asc-Ca-DCA) of Preparation Example 1 was administered to the mice for a total of 21 days. The changes in weight were analyzed, and the results are summarized in FIGS. 49A and 49B.

In FIG. 49, each group represents the following. G1: normal control group, G2: the a vehicle (excipient) administration group, G3: megestrol administration group (125 mpk), G4: calcium salt of Preparation Example 1 oral administration group (200 mpk), G5: the calcium salt of Preparation Example 1 (100 mpk) intravenous administration group, and G6: the calcium salt of Preparation Example 1 (200 mpk) intravenous administration group.

Referring to FIG. 49, it was found that the weight loss rate of the vehicle control group G2 compared to the normal control group G1 on the 17th day was about 8.4% and about 13.3% on the 20th day (p<0.01 or p<0.0001). In the case of G6, it was found that the weight increased by about 6% on the 17th day and 13% on the 20th day compared to G2 (p<0.05).

### Experimental Example 12-5. Histopathological Analysis of Mouse Muscle Tissue by Treatment with Calcium Salt of Preparation Example 1

A CT26 mouse colorectal cancer cell line was subcutaneously transplanted into Balb/c mice to construct a syngeneic model, and the calcium salt (Asc-Ca-DCA) of Preparation

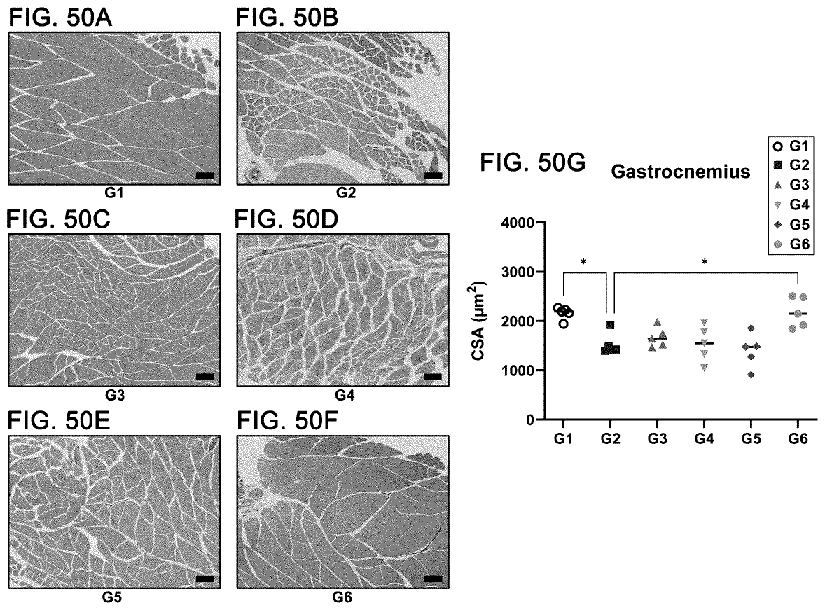

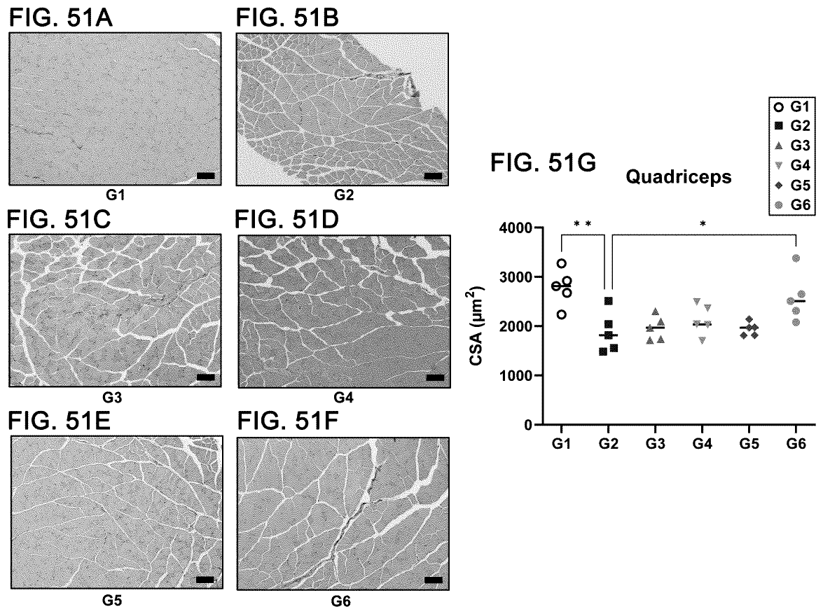

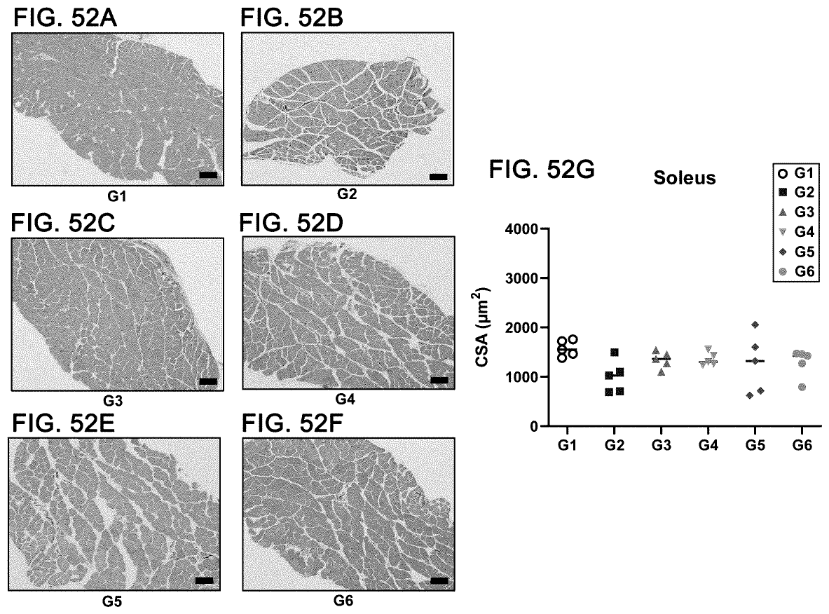

Example 1 was administered to the mice for a total of 21 days. The mice were sacrificed, and three types of skeletal muscle (gastrocnemius, quadriceps, and soleus) were extracted at autopsy, and histopathological analysis was performed. Cross-sectional images of muscle tissue were obtained, and the muscle fiber cross-sectional area (CSA) was measured, and the results are summarized in FIGS. 50 to 52.

In FIGS. 50 to 52, each group represents the following. G1: normal control group, G2: the a vehicle (excipient) administration group, G3: megestrol administration group (125 mpk), G4: calcium salt of Preparation Example 1 oral administration group (200 mpk), G5: the calcium salt of Preparation Example 1 (100 mpk) intravenous administration group, and G6: the calcium salt of Preparation Example 1 (200 mpk) intravenous administration group. Scale bars inserted in A to F of FIGS. 50 to 52 represent a horizontal length of 100 μm.

Referring to FIGS. 50 to 52, it was confirmed that G4 to G6 treated with the calcium salt of Preparation Example 1 increased the cross-sectional area of muscle tissue compared to the vehicle control group G2, and showed a level similar to that of group G3 treated with megestrol, which is a therapeutic agent for cachexia.

### Experimental Example 12-6. Evaluational Analysis of Mouse Behavior by Treatment with Calcium Salt of Preparation Example 1

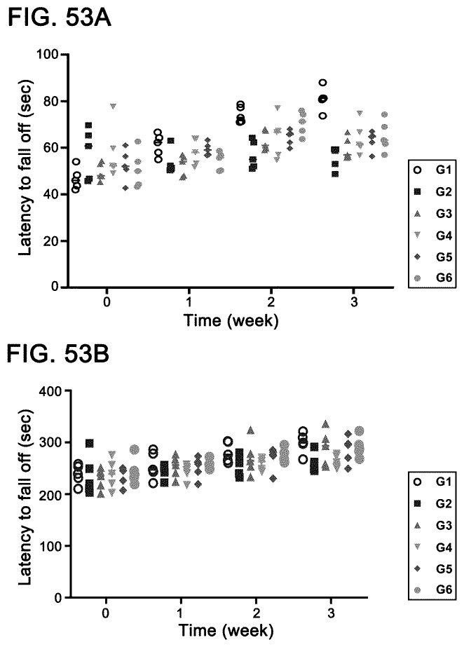

A CT26 mouse colorectal cancer cell line was subcutaneously transplanted into Balb/c mice to construct a syngeneic model, and the Rotarod latency test was performed once a week before and after administration of the test substance. After the animal was carefully placed on a Rotarod-treadmill (JD-A-07RA5, B.S Technolab Inc., Korea), and the measurement was made for 300 seconds while increasing the rotation speed from 4 rpm to 40 rpm at regular intervals and, and the results are summarized in FIG. 53A.

Along with the above, a wire hanging test was performed once a week, and the animal was placed on top of a wire cage, and then turned the cage upside down and hung over the home cage, and the waiting time until the animal fell was recorded and summarized in FIG. 53B.

Referring to FIG. 53A, it was confirmed in the Rotarod-treadmill test that the vehicle control group G2 showed a decrease of about 23% compared to the normal control group G1, whereas G6 showed about 24% improvement compared to G2.

Referring to FIG. 53B, it was confirmed in the wire hanging test that G4 to G6 showed 5% to 10% improved values compared to the vehicle control group G2, while showing a level similar to that of G3 administered with megestrol, which is a positive control substance.

What is claimed is:

1. A pharmaceutical composition for preventing or treating cachexia and muscle loss comprising an ionic compound,
wherein the ionic compound comprises a calcium cation, a first anion, and a second anion, and
wherein the first anion and the second anion are different from each other, and are each independently anions of ascorbic acid, dichloroacetic acid, a branched-chain amino acid (BCAA), or derivatives thereof.

2. The pharmaceutical composition of claim 1, wherein the branched-chain amino acid is one or more selected from the group consisting of leucine, isoleucine, and valine.

3. The pharmaceutical composition of claim 1, wherein the ionic compound is administered at a dose of 20 mg/kg/day to 4,800 mg/kg/day.

4. The pharmaceutical composition of claim 1, wherein the pharmaceutical composition further comprises a pharmaceutically acceptable aqueous solvent, and wherein the degree of dissociation of the ionic compound is 1% to 50%.

5. The pharmaceutical composition of claim 4, wherein the pharmaceutical composition further comprises one or more active agents selected from the group consisting of appetite enhancers, anticancer agents, anti-inflammatory agents, antibacterial agents, antifungal agents, antiviral agents, immunomodulators, steroids, anticoagulants, anticonvulsants, antidepressants, antioxidants, and vitamins.

6. The pharmaceutical composition of claim 1, wherein the pharmaceutical composition further comprises one or more selected from pharmaceutically acceptable carriers, excipients, and diluents.

7. The pharmaceutical composition of claim 1, wherein the pharmaceutical composition is formulated into a liquid, powder, an agent for oral administration, an injection, an infusion, an aerosol, a tablet, a capsule, a pill, a depot, or a suppository.

8. The pharmaceutical composition of claim 1, wherein the pharmaceutical composition is administered orally, intravenously, subcutaneously, intramuscularly, or transmucosally.

9. The pharmaceutical composition of claim 1, wherein the cachexia and muscle loss are caused by cancer, tuberculosis, diabetes, AIDS, chronic obstructive pulmonary disease, multiple sclerosis, congestive heart failure, hemophilia, hypopituitarism, or liver cirrhosis.

10. The pharmaceutical composition of claim 9, wherein the cancer is selected from the group consisting of lung cancer, breast cancer, colorectal cancer, stomach cancer, liver cancer, brain cancer, pancreatic cancer, thyroid cancer, skin cancer, bone marrow cancer, lymphoma, uterine cancer, cervical cancer, ovarian cancer, kidney cancer, and melanoma.

11. The pharmaceutical composition of claim 1, wherein the muscle loss is caused by sarcopenia, atony, muscular atrophy, muscular dystrophy, amyotrophic lateral sclerosis, or myasthenia gravis.

12. A food composition comprising an ionic compound,
wherein the ionic compound comprises a calcium cation, a first anion, and a second anion, and
wherein the first anion and the second anion are different from each other, and are each independently anions of ascorbic acid, dichloroacetic acid, a branched-chain amino acid (BCAA), or derivatives thereof.

13. The food composition of claim 12, wherein the food composition is a health functional food.
